(19) United States
(12) Patent Application Publication
(10) Pub. No.: US 2025/0152928 A1
(43) Pub. Date: May 15, 2025

(54) OINTMENT CAPSULE AND DISPENSING SYSTEM

(71) Applicant: Michael Hirschl, Holon (IL)

(72) Inventor: Michael Hirschl, Holon (IL)

(21) Appl. No.: 18/834,639

(22) PCT Filed: Feb. 26, 2023
(86) PCT No.: PCT/IL2023/050200

§ 371 Date: Jul. 31, 2024

Related U.S. Application Data
(60) Provisional application No. 63/324,649, filed on Mar. 29, 2022.

(51) Int. Cl. A61M 35/00; A45D 19/02; A45D 34/00; A45D 34/04; A45D 40/26; A61C 19/06

(52) U.S. Cl. CPC A61M 35/003; A45D 19/02; A45D 34/04; A45D 40/26; A61C 19/066; A45D 2034/005; A61M 2205/6054; A61M 2205/6072

(57) ABSTRACT

An aspect of some embodiments of the current invention relates to a capsule which may be used to dispense an ointment to provide the user with a pre-determined amount of the ointment, thereby reducing waste and/or providing the required dose for effective treatment and/or application of the ointment. Additionally, such a capsule may be refilled and/or recycled. In addition, the capsule may include a verifiable identification tag. A further aspect of the current invention is a dispenser configured to dispense the ointment from a capsule. The dispenser may include a means of activating the ointment, for example a light of a particular frequency, such as, ultraviolet light, infrared light, etc., a heater, for local heating, ultrasound, etc., and/or a combination thereof.

### BACKGROUND OF THE INVENTION

The current invention, in some embodiments thereof, relates to an ointment capsule and preferably, an ointment dispensing system.

The global cosmetics market size has been valued at $380.2 billion in 2019, and is projected to reach $463.5 billion by 2027. Additionally, the global topical drug delivery market is projected to reach $317.8 billion by 2027. However, most people applying cosmetics and/or medication topically do not do so efficiently, either applying too much or too little of the product. Additionally, a large portion of the beauty industry's waste comes from packaging.

Therefore, there is a need for means of providing the correct amount or dose of a cosmetic product or medication to a user for topical administration, which may be recycled or reused, thereby reducing waste.

### SUMMARY

According to an aspect of some embodiments of the invention, there is provided an ointment capsule including: an identification tag; at least one nozzle attached to or integrated with a capsule top; the capsule top sealed to a capsule base; and at least one punch aligned with the at least one nozzle, wherein the punch is configured to penetrate the capsule top and dispense the ointment through the nozzle when pressure is applied to the capsule.

According to some embodiments of the invention, the capsule is round, cylindrical, bowl shaped, circular, book shaped, accordion shaped, bottle shaped, square shaped, cubical, or pyramidal in shape.

According to some embodiments of the invention, a cross section of the capsule is round, oval, square, rectangular, triangular, pentagonal or hexagonal.

According to some embodiments of the invention, a width of the capsule ranges between about 1 mm to about 5 cm.

According to some embodiments of the invention, a volume of a capsule holder of a dispenser configured to hold the capsule may range between about 1 ml to about 100 ml.

According to some embodiments of the invention, the nozzle has a length of between about 1 mm to about 1 cm.

According to some embodiments of the invention, the punch has a sharp tip.

According to some embodiments of the invention, the punch does not completely occlude the nozzle on penetration of the capsule top.

According to some embodiments of the invention, the capsule includes a screw.

According to some embodiments of the invention, the identification tag is selected from the group consisting of a barcode, QR code, RFID, magnetic code, or a readable chip.

According to an aspect of some embodiments of the invention, there is provided an ointment dispenser including: a dispenser head, wherein the dispenser head including at least one nozzle opening and at least one activator; a capsule holder fitted within a dispenser body, wherein the dispenser head and the dispenser body are connected to each other; and a driver located within the dispenser body configured to push the capsule holder against an inner surface of the dispenser head.

According to some embodiments of the invention, the dispenser head further includes at least one vent.

According to some embodiments of the invention, the dispenser head further includes at least one guide light.

According to some embodiments of the invention, the at least one activator is selected from a group consisting of a light of a particular frequency, a heater, ultrasound, and a combination thereof.

According to some embodiments of the invention, the dispenser head is further configured to open and close.

According to some embodiments of the invention, the dispenser head is further configured to lock in a closed configuration.

According to some embodiments of the invention, the dispenser head further includes a slot into which a capsule is inserted.

According to some embodiments of the invention, the dispenser body further includes a user interface.

According to some embodiments of the invention, the dispenser body further includes a rechargeable battery and a battery housing.

According to some embodiments of the invention, the dispenser body further includes a charging port on or within a base of the dispenser body.

According to some embodiments of the invention, the charging port connects to a charging station.

According to some embodiments of the invention, the driver includes a piston arm connected through a transmission to a lead screw and a ball-screw arm attached to a slide track on a plunger.

According to some embodiments of the invention, the dispenser further includes an identification tag reader.

According to some embodiments of the invention, the identification tag reader is configured to verify a veracity of a capsule inserted into the dispenser.

According to an aspect of some embodiments of the invention, there is provided a method for dispensing an ointment from an ointment capsule using an ointment dispenser, the method including: inserting an ointment capsule into a capsule holder in an ointment dispenser; verifying the capsule; triggering a driver in a body of the dispenser to compress the verified capsule; pushing a punch through a capsule top; and squeezing the ointment out of a nozzle of the verified capsule through a head of the dispenser.

According to some embodiments of the invention, the verifying includes reading an identification tag on the capsule and verifying the read identification tag using an internal database.

According to some embodiments of the invention, the verifying includes reading an identification tag on the capsule and verifying the read identification tag by connecting to an on-line database over a wireless network.

According to some embodiments of the invention, the method further includes activating the ointment using at least one activator on the head of the dispenser, wherein the at least one activator is selected from a group consisting of a light of a particular frequency, a heater, ultrasound, and a combination thereof.

According to some embodiments of the invention, the activating takes place before, during or after application of the ointment.

### DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1: Perspective view of an unused round capsule in accordance with some embodiments of the current invention.

FIG. 2: Side view of a used round capsule in accordance with some embodiments of the current invention.

FIG. 3: Perspective view of a round capsule top in accordance with some embodiments of the current invention.

FIG. 4: Perspective view of a round capsule bottom in accordance with some embodiments of the current invention.

FIG. 5: Side view of a nozzle in accordance with some embodiments of the current invention.

FIG. 6: Perspective view of a punch in accordance with some embodiments of the current invention.

FIG. 7A-D: Cut away views of various punches in accordance with some embodiments of the current invention.

FIG. 8: Exploded perspective view of a round capsule in accordance with some embodiments of the current invention.

FIG. 9: Perspective view of a multi-nozzle capsule in accordance with some embodiments of the current invention.

FIG. 10: Perspective view of a single nozzle capsule in accordance with some embodiments of the current invention.

FIG. 11: Cut away view of an integrated capsule in accordance with some embodiments of the current invention.

FIG. 12: Perspective view of an integrated capsule in accordance with some embodiments of the current invention.

FIG. 13: Cut away view of a book capsule in accordance with some embodiments of the current invention.

FIG. 14: Perspective view of a used book capsule in accordance with some embodiments of the current invention.

FIG. 15: Side view of a screw capsule in accordance with some embodiments of the current invention.

FIG. 16: Cut away view of a screw capsule in accordance with some embodiments of the current invention.

FIG. 17: Exploded perspective view of a round capsule in accordance with some embodiments of the current invention.

FIG. 18: Side view of an accordion capsule in accordance with some embodiments of the current invention.

FIG. 19: Perspective view of an accordion capsule in accordance with some embodiments of the current invention.

FIG. 20A-B: Side views of a dispenser in accordance with some embodiments of the current invention.

FIG. 21: Back view of a dispenser in accordance with some embodiments of the current invention.

FIG. 22: Front view of a dispenser in accordance with some embodiments of the current invention.

FIG. 23: Perspective view of a single nozzle dispenser in accordance with some embodiments of the current invention.

FIG. 24: Perspective view of a multi-nozzle dispenser in accordance with some embodiments of the current invention.

FIG. 25A-B: Perspective views of a smooth faced dispenser in accordance with some embodiments of the current invention.

FIG. 26: Perspective view of a dispenser and charging station in accordance with some embodiments of the current invention.

FIG. 27: Back view of a dispenser and charging station in accordance with some embodiments of the current invention.

FIG. 28: Perspective view of a charging station in accordance with some embodiments of the current invention.

FIG. 29: Cut away view of a dispenser in accordance with some embodiments of the current invention.

FIG. 30: Cut away view of a dispenser in accordance with some embodiments of the current invention.

FIG. 31: Perspective view of an open side loading dispenser with a capsule in accordance with some embodiments of the current invention.

FIG. 32: Perspective view of an open side loading dispenser with a capsule in accordance with some embodiments of the current invention.

FIG. 33: Perspective view of an open dispenser with a capsule in accordance with some embodiments of the current invention.

FIG. 34: Cut away view of an open dispenser with a round capsule in accordance with some embodiments of the current invention.

FIG. 35: Cut away view of an open dispenser with a book capsule in accordance with some embodiments of the current invention.

FIG. 36: Cut away view of an open dispenser with a screw capsule in accordance with some embodiments of the current invention.

FIG. 37: Side view of a driver and capsule in accordance with some embodiments of the current invention.

FIG. 38: Exploded view of a capsule and dispenser in accordance with some embodiments of the current invention.

FIG. 39: Exploded view of a capsule and dispenser in accordance with some embodiments of the current invention.

FIG. 40: Block diagram of a capsule in accordance with some embodiments of the current invention.

FIG. 41: Block diagram of a dispenser in accordance with some embodiments of the current invention.

FIG. 42: Block diagram of a capsule in a dispenser in accordance with some embodiments of the current invention.

FIG. 43: A flow diagram describing the use of the capsule in accordance with some embodiments of the current invention.

FIG. 44: A flow diagram describing the use of the dispenser in accordance with some embodiments of the current invention.

FIG. 45: A flow diagram describing the use of the dispenser in accordance with some embodiments of the current invention.

### DESCRIPTION OF THE INVENTION

The present invention, in some embodiments thereof, relates to an ointment capsule and preferably, an ointment dispensing system.

### Overview

An aspect of some embodiments of the current invention relates to a capsule which may be used to dispense an ointment, such as a skin cream e.g., a serum, moisturizer, cleanser, shaving cream, sunscreen, mosquito repellent, etc., a cosmetic product such as foundation, concealer, primer, liquid makeup, skin blemish treatment, etc., and/or a medication such as a topical medication, for example for treatment of a medical condition such as burns, scars, acne, rosea, eczema, a fungal, bacterial or viral infection, etc., and/or a dental treatment, such as tooth whitener, sealant, etc., and/or a hair treatment, e.g., for the application of hair dye, hair repair serum, conditioner, color fixer, hair extensions, scalp treatment, etc., and/or a nail treatment, e.g., a nail treatment e.g., to encourage growth and/or strengthen the nails, a primer, e.g., prior to application of a nail polish, nail adhesive, gel, etc. and, optionally, a dispenser thereof, is disclosed and described.

As used herein, the term “ointment” refers to a preparation for external use (e.g., applied to skin, hair, teeth, nails, etc.) for medicinal purposes or as a cosmetic. Optionally, the ointment may be an oil and/or water-based liquid or gel. Optionally, the ointment may be viscous.

Advantageously, such a capsule may provide the user with a pre-determined amount of the ointment, thereby reducing waste and/or providing the required dose for effective treatment and/or application of the ointment. Alternatively, and/or additionally, such a capsule may be refilled and/or recycled.

According to some embodiments, the capsule may be filled by the user prior to dispensing and/or pre-filled with the required ointment at a manufacturing site, such as a factory, pharmacy, dispensary, etc. Optionally, the capsule may comprise a capsule bottom, a capsule top or lid which may seals the capsule, one or more nozzles on the upper surface of the capsule top and/or a punch or screw. Optionally, the punch may be used to perforate the capsule top. Optionally, the punch may be attached to and/or may be an integral part of the capsule bottom. Optionally, the perforated capsule top may allow application of the ointment through the one or more nozzles on squeezing of the capsule. Optionally, the one or more nozzles may be attached to the upper surface of the capsule top and/or the one or more nozzles may be an integral part of the capsule top. Optionally, the capsule may be of a variety of sizes and/or shapes depending, for example, on the required quantity of ointment, the ointments viscosity, its case of spread, and/or the required dose. For example, the capsule may be circular, bowl shaped, bottle shaped, accordion shaped, book shaped, etc. Optionally, the capsule may include a screw. Optionally, the capsule may protect the ointment from light, oxidation, degradation, etc., prior to use. For the sake of this disclosure, the words top and proximal will mean the side of the dispenser that is placed against the skin and/or from which the material (e.g., ointment) is released. For the sake of this disclosure, the words bottom and distal will mean the side of the dispenser that is opposite the side placed against the skin and/or from which the material (e.g., ointment) is released.

According to some embodiments, the capsule may have an identification tag. Optionally, the identification tag may allow the user to identify the contents of the capsule. Optionally, the identification tag may allow a dispenser to determine of the capsule is genuine and/or contains the required ointment and/or the quantity of ointment contained therein. Optionally, the identification tag may be a barcode, QR code, RFID, etc. Optionally, the identification tag may be electronic. Optionally, the dispenser may send a signal to and/or receive a signal from a wireless device such as a cellular phone, laptop, wireless network on reading the identification tag. Optionally, the dispenser may not function unless the identification tag is verified.

According to some embodiments, a dispenser may be used to dispense the ointment from a capsule. Optionally, the dispenser may comprise a body and a head. Optionally, the head may be opened (e.g., from the top, side, base, etc.) and/or may include an opening and/or slot into which a capsule may be placed. Optionally, the head may be connected to the body, for example by a hinge. Optionally, the head may be locked onto the body. Optionally, the dispenser head and/or body may include a capsule holder. Optionally, the capsule holder may accept a variety of capsule sizes and/or depending on the required quantity of ointment, the ointments viscosity, its ease of spread, and/or the required dose. For example, the capsule may be circular, bowl shaped, bottle shaped, accordion shaped, book shaped, etc.

According to some embodiments, the dispenser may include a means of activating the ointment, for example a light of a particular frequency, such as, ultraviolet light, infrared light, etc., a heater, for local heating, ultrasound, etc., and/or a combination thereof. Optionally, the ointment may be activated by the dispenser prior to dispensing. Optionally, the ointment may be activated concurrently to dispensing from the dispenser. Optionally, the ointment may be activated a short time after dispensing either by the dispenser and/or by an additional device.

Alternatively, and/or additionally, the dispenser may be designed for a specific sized and/or shaped capsule. Optionally, the head may comprise an opening for dispensing ointment from the capsule. Optionally, the size of the head opening and/or nozzle opening may be adjusted depending on the user's requirements. Optionally, the head and/or body may comprise a seal which may prevent entrance of ointment into the mechanism of the dispenser from an opened and/or used capsule. For example, the system may be designed to dispense the cream directly from the capsule to the skin (or another external surface) without the cream touching the dispenser. Optionally, the dispenser may be easily cleaned in order to allow use of multiple types of capsules without cross contamination, such as, from ointment adhering to the dispenser. Optionally, the inside, outside, or both of all parts of the dispenser may be waterproof. Optionally, the inside, outside, or both of all parts of the dispenser may be easy to clean.

According to some embodiments, the dispenser head may comprise a means of activating the ointment, such as one or more lights of a selected frequency (e.g., UV, IR, etc.), one or more heaters (e.g., heating pads, flex heating elements, etc.), one or more additional activators (e.g., ultrasound, massage pads, etc.) and/or combinations thereof. Optionally, the dispenser head may comprise one or more vents to release pressure and/or heat. Optionally, the one or more activators may be connected to a power button on the body of the dispenser. Optionally, the capsule may be pretreated by the dispenser head prior to use. Optionally, an external surface of the user (e.g., skin, teeth, hair, nails, etc.) may be activated by one or more activators on the dispenser head prior to, during and/or after application of the ointment.

According to some embodiments, the dispenser body may comprise an driver to drive expulsion of material from the capsule. Optionally, the driver may include an actuator. Optionally, the driver may include a plunger and/or an actuator. Optionally, the driver may include a motor and/or a drive screw and/or a ball-screw arm and/or a slider. Optionally, the plunger may include a slider track, Optionally, the dispenser may include one or more batteries, a power button, a switch connected to the driver, etc. Optionally, the dispenser may be portable. Optionally, the dispenser may be designed to fit comfortably into a user's hand. Optionally, the dispenser may be wireless. Optionally, the driver may be activated manually and/or electronically. Optionally, the driver may comprise a piston arm. Optionally, activation of the driver may push the bottom of the capsule towards the top of the capsule, such that a punch pierces the capsule top. Optionally, activation of the piston may squeeze the ointment out of the capsule through the nozzle.

According to some embodiments, dispensing from the capsule may be actuated by means of a screw drive (e.g., driven by a motor and/or manual twisting) and/or a linear actuator and/or a piston and/or gas pressure and/or a hydraulic actuator and/or manual force (e.g., pushing and/or squeezing by the user). Optionally the drive system may be continuous. Alternatively, and/or additionally, the drive may be reset e.g., when a capsule is inserted, between each use, etc. For example, reset may be automatic (e.g., when the head and/or a section thereof is opened and/or when the drive has fully dispensed the cream, and/or when a capsule is removed after use, etc.). Alternatively, and/or additionally, the drive may be reset by the user (e.g., by activating reset mechanism e.g., by pushing a reset button). Alternatively, and/or addition reset may be manual (e.g., by manually returning an actuator to its reset position and/or turning back a screw etc.).

According to some embodiments, one or more batteries may be housed in a battery housing. Optionally, the battery may be rechargeable. Optionally, the base of the dispenser body may comprise a port for connecting it to a charging station. Optionally, the charging station may be connected by a port and/or socket to an electricity source, such as an AC or DC power source, power sources (e.g., 110 AC, 220 AC, 24V DC (e.g., a car battery, photovoltaic cell, etc.) and/or with different kinds of plugs and/or sockets and/or combinations thereof. Optionally, the dispenser and/or charging station may contain one or more integrated wireless chargers.

According to some embodiments, the dispenser may comprise an identification tag reader and/or scanner. Optionally, the dispenser may comprise a wireless transmitter. Optionally, the dispenser may connect to a wireless network to verify an identification tag. Optionally, the wireless network may be a cellular network, a satellite network and/or a local network. Optionally, verification may be made by matching the identification tag to an identifier in a local database (e.g., within the dispenser, in an app, etc.), and/or an off-site database e.g., a cloud, etc., belonging to a capsule manufacturer, the dispenser manufacture and/or public domain. Optionally, the dispenser may connect to an app on a user's portable electronic device, such as cell phone, tablet, and/or laptop, in order to verify the identification tag, e.g., wirelessly by wi-fi, Bluetooth, etc. Optionally, the tag and reader may include an optical (for example, a bar code and reader and/or a QR code and reader and/or a human readable tag and a reader (for example a scanner and a text recognition routine)). Alternatively, and/or additionally, the tag and reader may be electronic (e.g., a RFID) and/or magnetic (e.g., a magnetic strip and reader etc.

According to some embodiments, the volume of the capsule may range between about 0.1 to about 1 ml, and/or between about 1 to about 5 ml, and/or between about 5 to about 10 ml, and/or between about 10 to about 100 ml, and/or between about 100 to about 200 ml, and/or between about 200 to about 500 ml, and/or between about 500 to about 1000 ml, and/or any sub-ranges therebetween.

According to some embodiments, the capsule may be cylindrical, bowl shaped, circular, book shaped, accordion shaped, bottle shaped, square shaped, cubical, pyramidal, etc. Optionally the cross section of the capsule may be round and/or oval and/or square and/or rectangular and/or pentagonal and/or hexagonal and/or another form. Optionally, the width and/or diameter of the capsule may range between about 1 mm to about 1 cm, and/or between about 1 to about 5 cm, and/or between about 5 to about 10 cm. Optionally, the length and/or depth of the capsule may range between about 1 mm to about 5 mm, and/or between about 5 mm to about 30 mm, and/or between about 3 cm to about 10 cm. Optionally the capsule may include a tube. Optionally the capsule may include a screw.

According to some embodiments, the capsule may include a nozzle. Optionally, the nozzle may have a length of between about 1 mm to about 5 mm, and/or between about 5 mm to about 1 cm, and/or between about 1 cm to about 5 cm. Optionally the nozzle may have a width and/or diameter of between about 1 mm to about 5 mm, and/or between about 5 mm to about 1 cm, and/or between about 1 cm to about 5 cm.

According to some embodiments, the volume of the dispenser may range between about 1 ml to about 10 ml, and/or about 10 ml to about 100 ml, and/or about 100 ml to about 200 ml, and/or about 200 ml to about 500 ml, and/or about 500 to about 1000 ml, and/or about 1000 ml to about 5000 ml. Optionally, the dispenser may be cylindrical, and/or curved. Optionally, the dispenser may fit easily into the palm of a user's hand. Optionally, the width and/or diameter of the dispenser may range between about 1 mm to about 1 cm, and/or between about 1 to about 5 cm, and/or between about 5 to about 10 cm, and/or between about 10 to about 30 cm. Optionally, the length of the dispenser may range between about 1 mm to about 5 mm, and/or between about 5 mm to about 30 mm, and/or between about 3 cm to about 10 cm, and/or between about 10 cm to about 30 cm. Optionally the head of the dispenser may have a length of between about 1 mm to about 10 mm, and/or between about 10 mm to about 4 cm, and/or between about 4 cm to about 10 cm and/or between about 10 cm to about 20 cm. Optionally the head may have a width of between about 1 mm to about 10 mm, and/or between about 10 mm to about 4 cm, and/or between about 4 cm to about 10 cm and/or between about 10 cm to about 20 cm. Optionally the cross section of the dispenser may be round and/or oval and/or square and/or rectangular and/or pentagonal and/or hexagonal and/or vary over its length and/or another form.

According to some embodiments, the dispenser may include settable attributes. For example, the temperature of dispensing and/or a light frequency and/or intensity may be adjusted (e.g., the light may activate the cream and/or have a direct therapeutic effect on the skin of a user etc.). Optionally, the settings may be automatically adjusted according to the label of the capsule. Alternatively, and/or additionally, the dispenser may include a built-in user interface (e.g., an adjusting knob, a multiple position switch, a slider, a touch screen, an indicator light, a view screen etc.). Alternatively, and/or additionally, the settings may be controlled remotely, e.g., wirelessly using a dedicated remote control and/or using a user's computing device (e.g., a smart phone, tablet, laptop, desktop computer, etc. and/or a dedicated application on the computing device).

In some embodiments, the device may include a LED and/or other light source that projects onto the skin and/or the ointment Infra-red, near infra-red, Blue, Red, White, Yellow, Green, Purple or other light). Alternatively or additionally, the device may include actuators for ATP therapy. Optionally, the device may include a laser (for example, for applying low level laser therapy (LLLT)). Alternatively or additionally, the device may perform BioPhoton Therapy and/or Microcurrent therapy.

According to some embodiments, the user may be a private individual, a cosmetologist and/or a beauty, medical or dental professional. Optionally, the system may allow multiple users to dispense a variety of different ointments from various capsules using the same dispenser. Optionally, a single user may use a variety of different ointments from a variety of capsules. Optionally, the dispenser and/or capsule system may prevent cross contamination of various ointments.

For example, a capsule bottom may be filled with an ointment, the capsule may then be sealed. The capsule may then be stored for an extended period without affecting the stability of the ointment. When required, a user may then load a capsule into a dispenser. The user may then turn on the dispenser and dispense the ointment directly onto the skin. The ointment may optionally be activated. The capsule may then be replaced by a capsule comprising a different ointment for a different use or for use on a different part of the body.

### Specific Embodiments

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

In an embodiment of the current invention relates to a capsule which may be used to dispense an ointment is disclosed and described. For example, the ointment may be a skin cream e.g., a serum, moisturizer, cleanser, shaving cream, sunscreen, mosquito repellent, etc., cosmetic product such as an anti-aging treatment, skin blemish treatment, cosmetic such as foundation, concealer, primer, liquid makeup, etc., and/or a medication such as a topical medication, for example for treatment of a medical condition such as burns, scars, acne, rosea, eczema, a fungal, bacterial or viral infection, etc., and/or a dental treatment, such as tooth whitener, sealant, etc., and/or a hair treatment, e.g., for the application of hair dye, hair repair serum, conditioner, color fixer, hair extensions, scalp treatment e.g., to stimulate hair growth, etc., and/or a nail treatment, e.g., a nail treatment e.g., to encourage growth and/or strengthen the nails, a primer, e.g., prior to application of a nail polish, a nail adhesive, a gel, etc.

According to some embodiments, the capsule may comprise two parts, such as, a bottom container and a top. Optionally, the capsule bottom and capsule top may be connected, e.g., by gluing, by mechanical sealing, etc.

According to some embodiments, the capsule bottom may comprise a lip. Optionally, the capsule bottom may comprise an identification tag. Optionally, a punch may be connected to, embedded on, and/or be manufactured as an integral part of the capsule bottom. Optionally, the punch may have a pointed and/or sharp tip. Optionally, the punch may be a sharp stake, a hollow needle, a finned projection and/or may have a variable width from its tip to its base.

According to some embodiments, the capsule top may comprise a lid. Optionally, the capsule top may be flat. Optionally, the capsule top may comprise a tight area for puncture. Optionally, the capsule top may comprise a neck. Optionally, one or more nozzles may be attached to the capsule top. Optionally, one or more nozzles may be attached over the capsule neck. Optionally, one or more nozzles may be connected to, embedded on, and/or be manufactured as an integral part of the capsule top. Optionally, the size of a nozzle opening may be adjusted to control the flow of the ointment. Optionally, a nozzle may be made from a plastic, flexible, and/or clastic material.

According to some embodiments, the capsule may be of a variety of sizes e.g., the length, width, depth and/or volume of the capsule may be varied. Optionally, the size of the required capsule may depend on the amount of the required ointment, e.g., depending on the use, such as full-face coverage, coverage of a selected area, or depending on the required dose of a medication, etc. For example, a capsule may include from 1 to 10 ml and/or between 10 to 50 ml. Optionally, the viscosity of the ointment may be adjusted to provide the required consistency for ease of dispensing. Optionally, the capsule bottom may be filled with an ointment and then sealed with a capsule top. Optionally, the capsule top may be filled with an ointment and then sealed with a capsule bottom. Optionally, the capsule may be circular, bowl shaped, bottle shaped, accordion shaped, book shaped, etc. Optionally, the capsule may protect the ointment from light, oxidation, degradation, etc., prior to use. Optionally, the capsule may maintain the stability of the ointment formulation. Optionally, the capsule may be filled by the user prior to use. Optionally, the capsule may be pre-filled with the required ointment at a manufacturing site, such as a factory, pharmacy, dispensary, etc. Optionally, each capsule may be single use. Optionally, each capsule may be opened, cleaned, optionally sterilized, refilled and resealed for multiple uses.

According to some embodiments, all or part of the capsule may be made from recycled and/or recyclable materials.

According to some embodiments, all or part of the capsule may be recyclable and/or reusable.

According to some embodiments, on squeezing and/or pushing the base of the capsule towards the top of the capsule, the punch may perforate the capsule top. Optionally, the perforated capsule top may allow application of the ointment through one or more nozzles on applying pressure to the capsule. Optionally, the capsule neck may be sealed towards the opening to prevent return flow of the ointment. Optionally, the capsule may include a screw. Optionally, rotation of the screw may force the ointment out of the capsule through a nozzle. Optionally, the screw may be rotated manually and/or automatically.

According to some embodiments, the user may be a private individual, a cosmetologist and/or a beauty, medical or dental professional. Optionally, the capsule may be used in a user's home and/or in a medical clinic and/or dental clinic and/or beauty salon.

According to some embodiments, the capsule may include an identification tag. Optionally, the identification tag may allow the user to identify the contents of the capsule, and/or the amount of the contents of the capsule. Optionally, the identification tag may allow determination of whether the capsule is genuine and/or contains the required ointment. Optionally, the identification tag may be a barcode, QR code, RFID, etc. Optionally, the identification tag may be electronic. Optionally, a user may scan the identification tag using a wireless electronic device, e.g., a cellular phone. tablet, laptop, barcode reader, specialized device, etc. Optionally, verification may be made by matching the identification tag to an identifier in a database. Optionally, the dispenser may verify an identification tag internally, e.g., using an internal database which may be periodically updated. Optionally, the dispenser may connect to a wireless network to connect to an on-line database to verify an identification tag. Optionally, the wireless network may be a cellular network, a satellite network and/or a local network. Optionally, verification may be made by matching the identification tag to an identifier in a local and/or off-site database e.g., a cloud, etc., belonging to a capsule manufacturer, dispenser manufacturer, government sponsored site and/or public domain. Optionally, verification may be made by an app on a user's computing device, such as cell phone, tablet, laptop and/or desktop computer, VR glasses, smartwatch, etc.

According to some embodiments, the current invention relates to a dispenser for dispensing ointment from a capsule is disclosed and described. Optionally, the dispenser may include a means of activating an ointment, for example, a light of a particular frequency such as ultraviolet light, infrared light, a heater for local heating, ultrasound, etc., a means of massaging and/or spreading the ointment, and/or a combination thereof. Optionally, the ointment may be activated by the dispenser prior to dispensing. Optionally, the ointment may be activated concurrently to dispensing from the dispenser. Optionally, the ointment may be activated a short time after dispensing by the dispenser and/or by an additional device.

According to some embodiments, the dispenser may comprise a body and a head. Optionally, the head may be opened and closed. Optionally, the head may include a slot for loading capsules. Optionally, the capsules may be loaded by inserting downward into the machine and/or upward and/or from any side and/or the back and/or the front. Optionally, the head may be connected to the body, for example by a hinge. Optionally, the head may be locked onto the body, e.g., by a clasp, catch, magnetic mechanism, mechanical mechanism, etc.

According to some embodiments, the dispenser head and/or body may include a capsule holder. Optionally, the capsule holder may accept a variety of capsule sizes and/or shapes depending on the required quantity of ointment, the ointments viscosity, its case of spread, and/or the required dose. Optionally, the capsule holder may be adjusted to accept various sizes and/or shapes of capsules. Optionally, the dispenser may be designed to prevent contact between the capsule contents and the inside of the dispenser, e.g., after punching the capsule, ointment may pass through the dispenser directly onto the user. Optionally, the dispenser may comprise a mechanism that may prevent return of the ointment once dispensed. Optionally, such a mechanism may prevent contamination of the inside of the dispenser by an ointment. Optionally, the head and/or body of the dispenser may comprise a seal which may prevent entrance of ointment into the mechanism of the dispenser from an opened and/or used and/or partially used capsule. Optionally, the dispenser may be easily cleaned. Optionally, the dispenser may allow use of multiple types of capsules without cross contamination, such as from ointment adhering to the dispenser. Optionally, the dispenser and capsule system may prevent cross contamination of the various ointments.

According to some embodiments, the dispenser head may comprise an opening for dispensing ointment from the capsule. Optionally, the size of the head opening may be adjusted depending on the user's requirements. Optionally, one or more nozzles of a capsule may extend through one or more openings located on the upper surface of the head. Optionally, this may ensure that the inside of the dispenser does not come into contact with the ointment.

According to some embodiments, the dispenser head may comprise a means of activating the ointment, such as one or more lights of a selected frequency (e.g., UV, IR, etc.), one or more heaters (e.g., heating pads, flex heating element, etc.), one or more additional activators (e.g., ultrasound, massage pads, etc.) and/or combinations thereof. Optionally, the dispenser head may comprise one or more vents to release excess pressure and/or heat. Optionally, the dispenser head may include one or more indicator lights to indicate when an activator is in use. Optionally, the one or more activators may be connected to a power button on the body of the dispenser.

According to some embodiments, the capsule may be pretreated by the dispenser head prior to use. Optionally, an external surface of the user may be activated by one or more activators on the dispenser head prior to application of the ointment. Optionally, an external surface of the user may be activated by one or more activators on the dispenser head during application of the ointment. Optionally, an external surface of the user may be activated by one or more activators on the dispenser head after application of the ointment. Optionally, an external surface of the user may be activated by an additional device. Optionally, one or more activators may be turned on, turned off, increased in intensity, decreased in intensity, etc. at any time during use of the dispenser.

According to some embodiments, the dispenser may be portable. Optionally, the dispenser may be designed to fit comfortably into a user's hand. Optionally, the dispenser may be wireless. Optionally, the dispenser body may comprise a driver, one or more internal batteries, a power button, a switch connected to the driver, etc. Optionally, the driver may be activated manually and/or electronically. Optionally, the driver may comprise a piston arm. Optionally, activation of the driver may push the bottom of the capsule such that the punch may pierce the capsule top. Optionally, activation of the piston may squeeze the ointment out of the capsule through one or more nozzles. Optionally, the driver may provide a steady and/or increasing or decreasing pressure to ensure even application of the ointment. Optionally, the pressure applied to the capsule by the driver may be adjusted prior to and/or during use. Optionally, the pressure applied to the capsule by the driver may be determined by the contents, size, shape and/or volume of the capsule.

According to some embodiments, one or more batteries may be housed in a battery housing. Optionally, the one or more batteries may be rechargeable. Optionally, the base of the dispenser body may comprise a port for connecting it to a charging station. Optionally, the charging station may be connected by a port and/or socket to an electricity source, such as an AC or DC power source, e.g., 110 AC, 220 AC, 24V DC (e.g., an electricity grid, a car battery, photovoltaic cell, etc.) and/or with different kinds of plugs and/or sockets and/or combinations thereof. Optionally, the dispenser and/or charging station may contain one or more integrated wireless chargers. Optionally, the port may be a USB port. Optionally, the charging station may be unique to the dispenser. Optionally, the charging station may be fixed to a surface, such as a table, floor, wall, etc. Optionally, the charging station may be portable.

According to some embodiments, the dispenser may comprise an identification tag reader and/or scanner, etc. wherein the identification tag may be a barcode, QR code, RFID, chip and electrodes, magnetic strip, and/or an electronic lock, e.g., similar to those used in printers to determine the veracity of ink cartridges, and/or means of completing an electric circuit, etc. Optionally, the dispenser may comprise a wireless transmitter. Optionally, verification may be made by matching the identification tag to an identifier in a database. Optionally, the dispenser may verify an identification tag internally, e.g., using an internal database which may be periodically updated. Optionally, the dispenser may connect to a wireless network to connect to an on-line database to verify an identification tag. Optionally, the wireless network may be a cellular network, a satellite network and/or a local network. Optionally, verification may be made by matching the identification tag to an identifier in a local and/or off-site database e.g., a cloud, etc., belonging to a capsule manufacturer, dispenser manufacturer, government sponsored site, public domain, etc. Optionally, verification may be required to prevent counterfeiting. Optionally, verification may be required to prevent mixing of incompatible substances. Optionally, verification may be required to ensure that the correct ointment is being applied to the correct user. Optionally, verification may be required to ensure that the capsules and/or contents are being used correctly. Optionally, the dispenser may connect to an app on a user's computing device, such as cell phone, tablet, laptop, desktop computer, smart phone, VR, etc. in order to verify the identification tag. Optionally, the dispenser may not function unless the identification tag is verified

According to some embodiments, the system may allow multiple users to dispense a variety of different ointments from various capsules using the same dispenser. Optionally, a single user may use a variety of different ointments from a variety of capsules using the same dispenser.

Reference is now made to the figures.

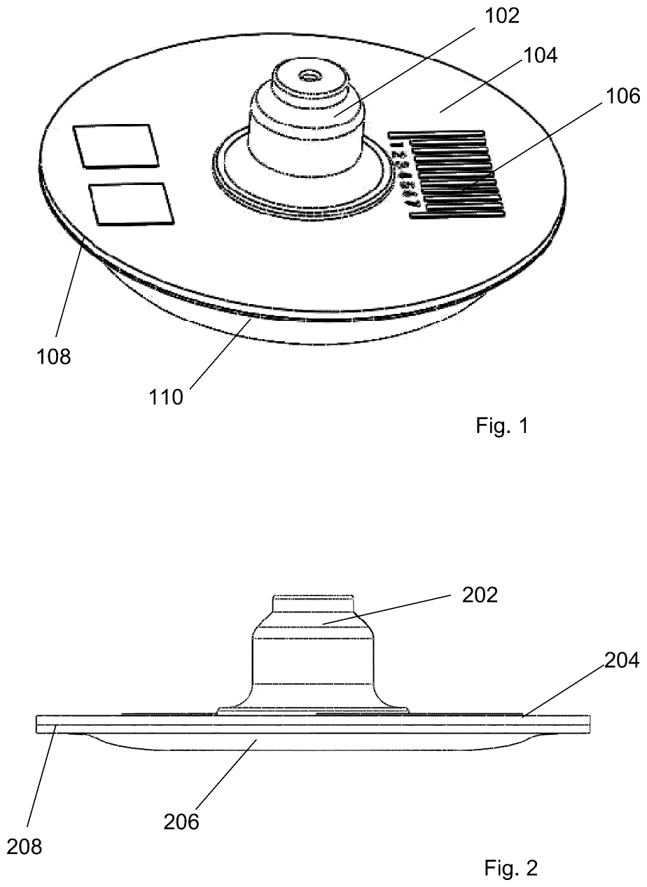

FIG. 1 is a perspective view of an unused round capsule in accordance with some embodiments of the current invention. For example, a round capsule may include, a top 104 with a nozzle 102. Optionally, a lip 108 of the top 104 connects between the top 104 and bottom 110 of the capsule. In some embodiments, a capsule includes an identification tag 106.

FIG. 2 is a side view of a used round capsule in accordance with some embodiments of the current invention. In FIG. 2, the capsule is illustrated after use, e.g., the capsule has been compressed releasing its contents. For example, a used round capsule may include, a top 204 with a nozzle 202 through which the ointment may have passed, with a lip 208 which may provide attachment between a capsule top 204 and bottom 206, wherein the capsule bottom 206 may be compressed towards the capsule top to dispense the ointment contained therein.

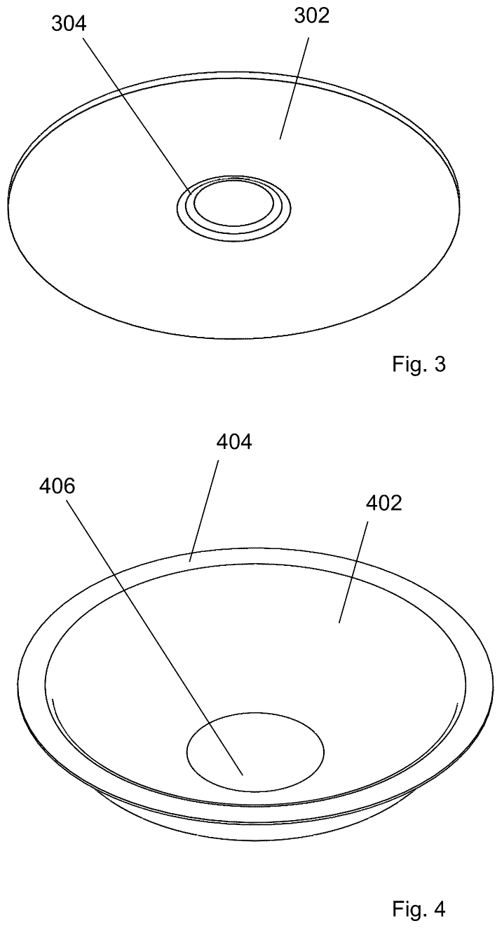

FIG. 3 is a perspective view of a round capsule top in accordance with some embodiments of the current invention. For example, the capsule top 302 may include a capsule neck 304.

FIG. 4 is a perspective view of a round capsule bottom in accordance with some embodiments of the current invention. For example, the capsule bottom 402 may include a lip 404 and an identification tag 406.

According to some embodiments, the capsule bottom and/or capsule top may include one or more elements, such as a printed figure, logo, name of the contents, manufacturing date, expiry date, etc. Optionally, the capsule bottom and/or capsule top may include a few words in braille e.g., on the top, base and/or sides.

According to some embodiments, the capsule bottom and capsule top may be made from different materials and/or may have different thicknesses in whole or in part. Optionally, the capsule top and capsule bottom may be made from high quality transparent, partially transparent, translucent and/or opaque plastic. Preferably, the plastic is non-toxic and stable when in contact with the ointment. For example, the capsule bottom may be made from aluminum foil, acrylic (polymethylmethacrylate), butyrate (cellulose acetate butyrate), polyvinyl chloride, polycarbonate, polyethylene terephthalate, glycol modified polyethylene terephthalate, polytetrafluoroethylene, polystyrene, polypropylene, polyamide, polyethylene, etc. Optionally, the capsule top may range in thickness between about 0.001 mm to about 0.01 mm, and/or about 0.01 mm to about 0.5 mm, and/or about 0.5 mm to about 1 mm, and/or about 1 mm to about 5 mm. Optionally, a portion of the capsule top may be made from a different and/or thinner material than the rest of the it, e.g., the capsule neck. Optionally, the capsule neck may be covered, sealed, partially covered, and/or open.

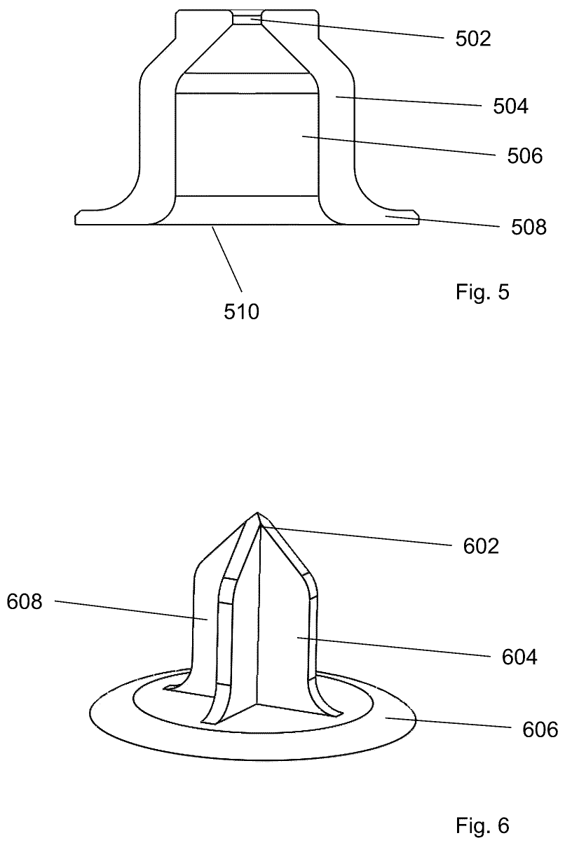

FIG. 5 is a cut away view of a schematic representation of a nozzle in accordance with an aspect of the current invention. For example, a nozzle 504 may include an opening 502, a hollow 506 (for example in its center), and a base 508. Optionally, the nozzle opening 502 may be adjustable. Optionally, the base 508 may be an integral part of and/or attached to the capsule top (not shown). Optionally, more than one nozzle may be attached to a capsule top. Optionally, a nozzle may be sealed. For example, there may be a seal 510 at the bottom of the hollow 506.

FIG. 6 is a perspective view of a schematic representation of a punch in accordance with an aspect of the current invention. For example, a punch 604 may include a point 602 and a base 606. Optionally, the base 606 may be an integral part of and/or attached to the capsule bottom (not shown). Optionally, more than one punch may be attached to a capsule bottom. Optionally, a punch may fit within a nozzle. Optionally, a punch may penetrate through a capsule neck. Optionally, a punch may penetrate through a capsule neck into the hollow space within a nozzle. Optionally, a punch may penetrate through a capsule neck into the hollow space within a nozzle and penetrate a seal on the nozzle opening. Optionally, a punch may not completely fill the hollow space within a nozzle. For example, when the punch is pressed into the space in the nozzle, it may leave channels 608 through which the contents of the capsule may pass out the nozzle. Optionally, the base 606 of the punch may be perforated, e.g., facilitating flow of the contents of the capsule to pass through base 606 when the punch is pushed fully into hollow 506. Alternatively or additionally, base 606 may include a closed surface that seals hollow 506 when the capsule has been fully squeezed releasing its contents.

According to some embodiments, the punch and/or the nozzle may be made from a metal or plastic. Preferably, the plastic is non-toxic and stable when in contact with the ointment. For example, the punch and/or the nozzle may be made from acrylic (polymethylmethacrylate), butyrate (cellulose acetate butyrate), polyvinyl chloride, polycarbonate, polyethylene terephthalate, glycol modified polyethylene terephthalate, polytetrafluoroethylene, polystyrene, polypropylene, polyamide, polyethylene, etc.

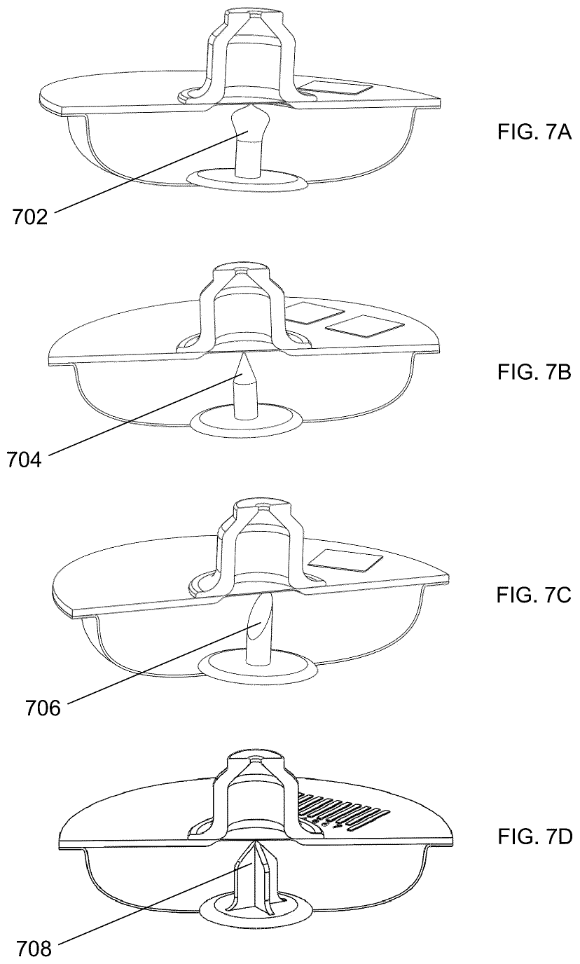

FIG. 7A-D: Cut away views of schematic representations of various punches in accordance with some embodiments of the current invention. For example, the punch may have a variable width, be a sharp stake 704, be needle shaped stake 706, and/or include one or more fins and/or leaves. Optionally, the punch may include a sharp tip (e.g., either having and beveled tip (e.g., stake 706) and/or a conical tip (e.g., stake 704) and/or a multi-leaved tip (e.g., stake 708) and/or a have a variable width tip (e.g., stake 702)) and may not completely occlude the base of the nozzle. Optionally, the punch may penetrate the nozzle base and/or the capsule top.

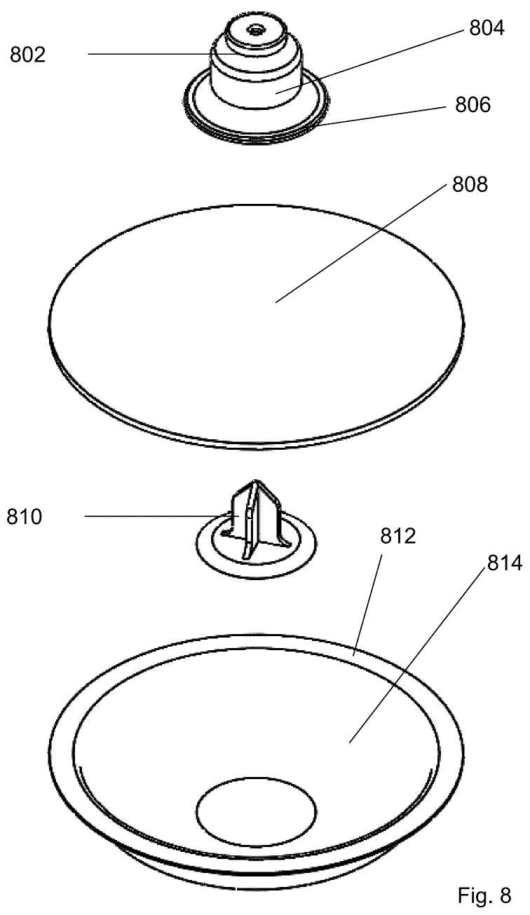

FIG. 8 is an exploded view of a schematic representations of a round capsule in accordance with an aspect of the current invention. For example, a nozzle 802, including a nozzle neck 804 and nozzle base 806, may be attached to or be an integral part of a capsule top 808 to which may be attached to a capsule bottom 814 at a lip 812. The capsule bottom 814 may include a punch 810 which may be attached to or may be an integral part of the capsule bottom. Optionally, the punch 810 may be located directly below the nozzle 802. Optionally, once the punch 810 has been pushed to perforate the capsule top 808, the punch 810 may not block the passage of the ointment through the nozzle 802. Optionally, the capsule top 808 and/or capsule bottom 814 may include an identification tag.

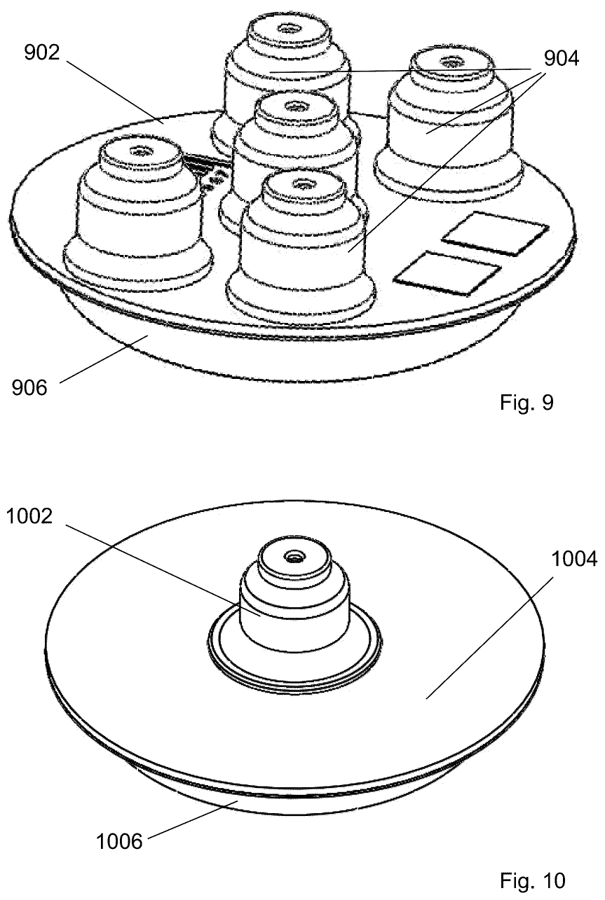

FIG. 9 is a perspective view of a multi-nozzle capsule in accordance with some embodiments of the current invention, and FIG. 10 is a perspective view of a single nozzle capsule in accordance with some embodiments of the current invention. For example, a capsule top 902, 1004, may include one or more nozzles 904, 1002. Optionally, each nozzle 904, 1002 may have a corresponding punch (not shown) on the capsule bottom 906, 1006. Optionally, use of more than one nozzle may provide rapid dispensing of an ointment, e.g., for an ointment which may be air, light, heat and/or time sensitive. For example, a capsule may have multiple small compartments, each compartment being openable and/or releasable separately. For example, one may use one compartment with a first nozzle and then another compartment with another nozzle. Optionally, the dispenser may be configured specifically for the multi-nozzle cartridge. For example, the dispenser may include separate openings for each nozzle. In some embodiment, each nozzle may include a separate seal and/or a separate mechanism (e.g., punch) to break the seal. Alternatively or additionally, the cartridge may include a main seal that is broken by one or more mechanisms (e.g., an single mechanism opens the seal so that material is released to all of the nozzles and/or multiple mechanisms open the seal for each nozzle). Optionally, a part of the dispenser may be replaceable. For example, a part of the dispenser head may change for single and/or multiple nozzle cartridges.

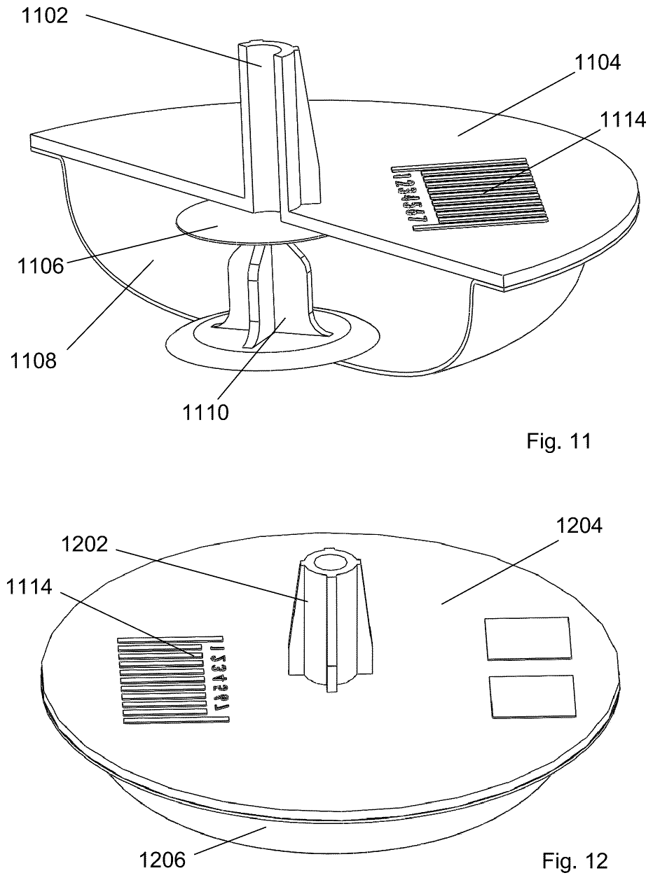

FIG. 11 and FIG. 12 are a cut away view and a perspective view, respectively of an integrated capsule in accordance with some embodiments of the current invention. For example, a nozzle 1102, 1202 may be integrated with a capsule top 1104, 1204. The capsule may include a seal 1106 which may completely or partially block and/or seal the base of the nozzle 1102. Optionally, the seal 1106 may be attached to the capsule top 1104. Optionally, a punch 1110 attached to a capsule bottom 1108, 1206 may penetrate the seal 1106 and allow passage of the ointment on dispensing. Optionally, the capsule may include an identification tag 1114. Optionally, tag 1114 may be printed directly on the capsule (from example on a top 1104 and/or flat surface thereof (e.g., near the nozzle).

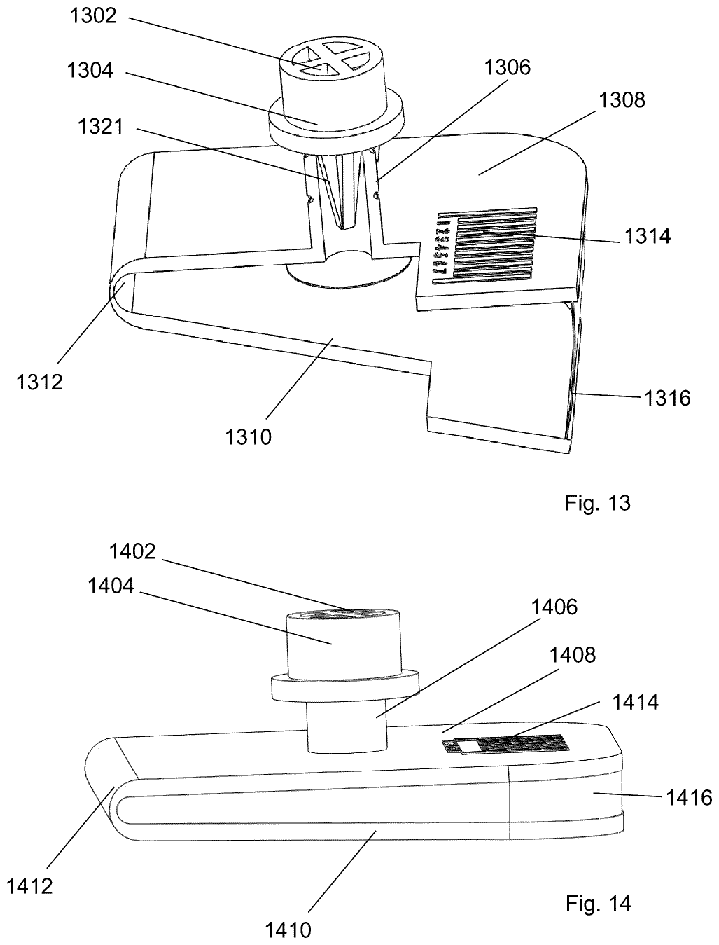

FIG. 13 and FIG. 14 are cut away and perspective views, respectively of an unused and used, respectively book capsule in accordance with some embodiments of the current invention. Optionally, a book capsule may collapse like a closing book as it releases material. For example, a top 1308, 1408 and bottom 1310, 1410 may approach each other by rotating around a fulcrum (e.g., side 1312, 1412). For example, the angle between the surface may be reduced as the capsule is being emptied. Optionally, the top 1308, 1408 and bottom 1310, 1410 may include rigid and/or flat plates. For example, one side 1312, 1412 of the capsule may pivot while the other side 1316, 1416 collapses. In some embodiments, the book capsule may include a nozzle 1304, 1404. Optionally nozzle 1304, 1404 includes an adjustable opening. For example, a punch 1321 may be tapered and/or be insertable to a differing degree to make the opening larger or smaller. Optionally, a capsule neck 1306, 1406 is attached to and/or integral with a capsule top 1308, 1408 and/or a capsule bottom 1310, 1410. One side 1316, 1416 of the book capsule may be larger than a side opposite to it 1312, 1412. Optionally, on application of pressure to the capsule, the ointment may be dispensed through the nozzle 1304, 1404 and the size of the sides 1312, 1316, 1412, 1416 may be equalized. Optionally, the book capsule may include an identification tag 1314, 1414.

In some embodiments (optionally with any type of capsule) a punch 1321 may be pushed through the seal of the capsule. Optionally, the punch may be external to the capsule. For example, the punch 1321 may be part of a nozzle opening that the user places on the nozzle before use. Alternatively or additionally, the nozzle opening 1402, 1302 may be permanently and/or movably connected to the capsule neck 1306. For example, when a user begins to use the capsule, he may push the nozzle opening 1402, 1302 towards and/or into the breaking the seal. Optionally, the punch 1321 will then be pushed into the capsule neck 1306 and/or break the seal.

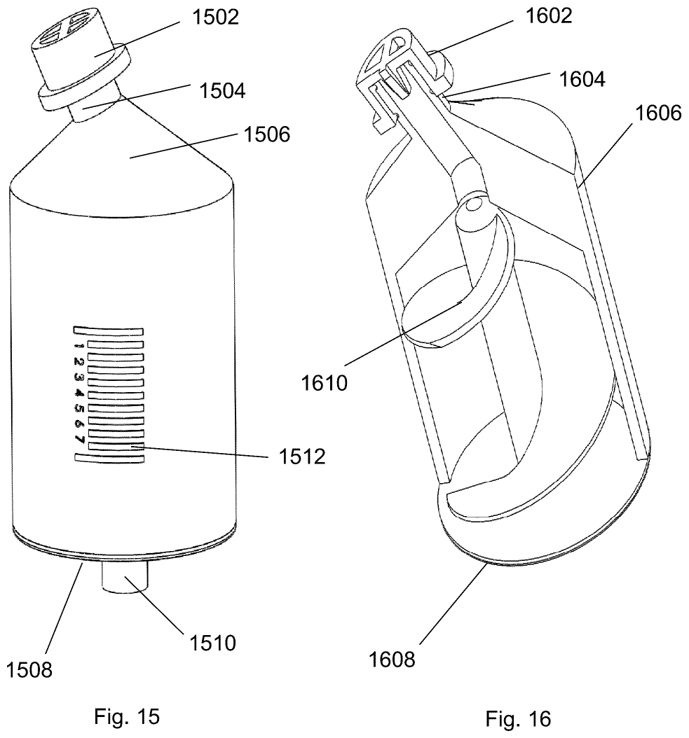

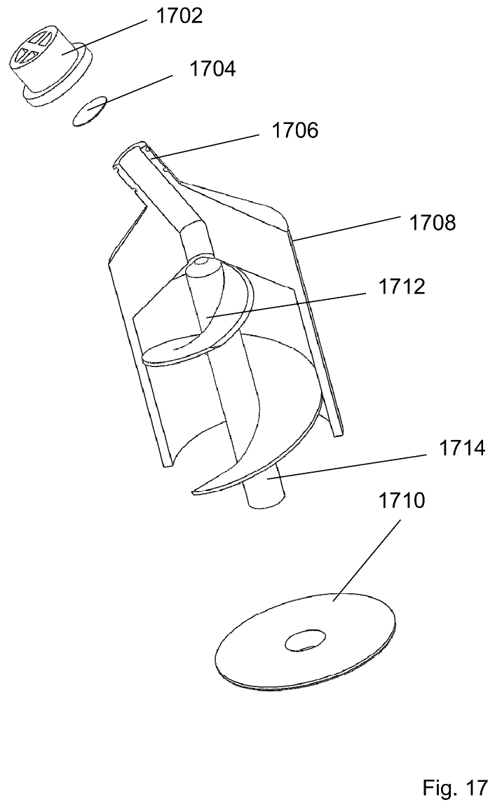

FIG. 15, FIG. 16, and FIG. 17 are a side view, cut away view and exploded view of a screw capsule in accordance with some embodiments of the current invention. For example, the screw capsule may include a nozzle 1502, 1602, 1702 attached to a capsule neck 1504, 1604, 1706 connected to a capsule top 1506, 1606, 1708 further connected to a capsule bottom 1508, 1608, 1710, wherein the capsule contains a screw 1610, 1712. Optionally, the nozzle includes a seal 1704. Optionally, a screw attachment 1510, 1714 may protrude through the capsule bottom 1508, 1608, 1710. Thus, the capsule bottom 1710 may include a bearing (e.g., a hole that allows an axle (e.g., screw attachment to protrude and rotate while supporting the axial position of the screw 1712. Optionally, the screw capsule may include an identification tag 1512, for example, printed on a side surface thereof. In some embodiments, a rotary driver in the dispenser will rotate a connector on the capsule that will rotate the screw 1610 of the capsule and/or drive the ointment into the nozzle.

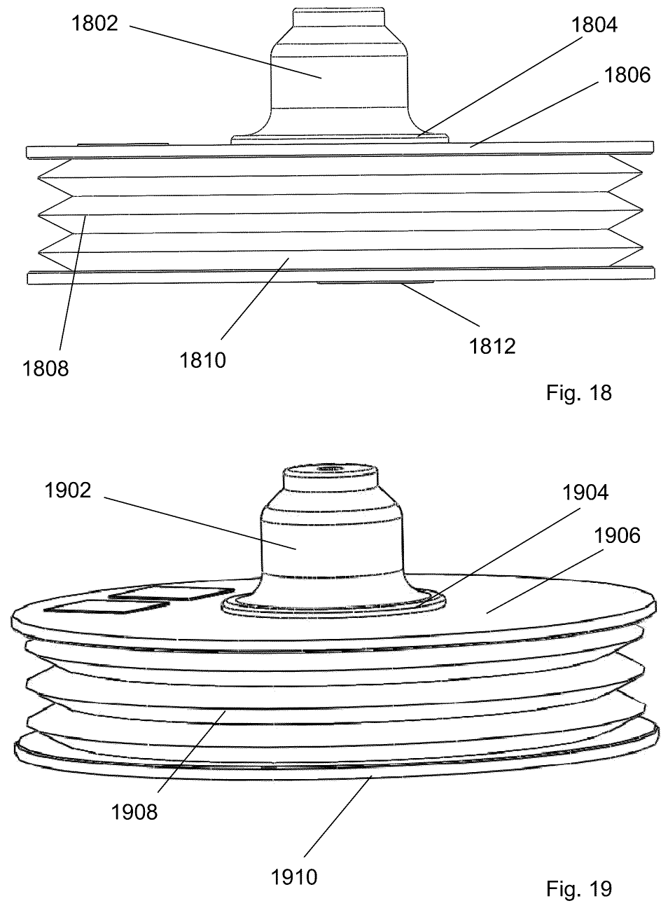

FIG. 18 and FIG. 19 are a side view and a perspective view, respectively of an accordion capsule in accordance with some embodiments of the current invention. For example, the accordion capsule may include a nozzle 1802, 1902, which may be attached by a capsule neck 1804, 1904 to a capsule top 1806, 1906. Optionally, the body and/or sides 1808, 1908 of the accordion capsule may be foldable. Optionally, the capsule bottom 1810, 1910 may include an identification tag 1812. Optionally, material is pushed out of the capsule by pushing the capsule bottom 1810, 1910 towards the capsule top 1806, 1906. Optionally, the at the bottom 1810, 1910 is pushed towards the top the sides 1908 collapse. Optionally, the sides collapse symmetrically.

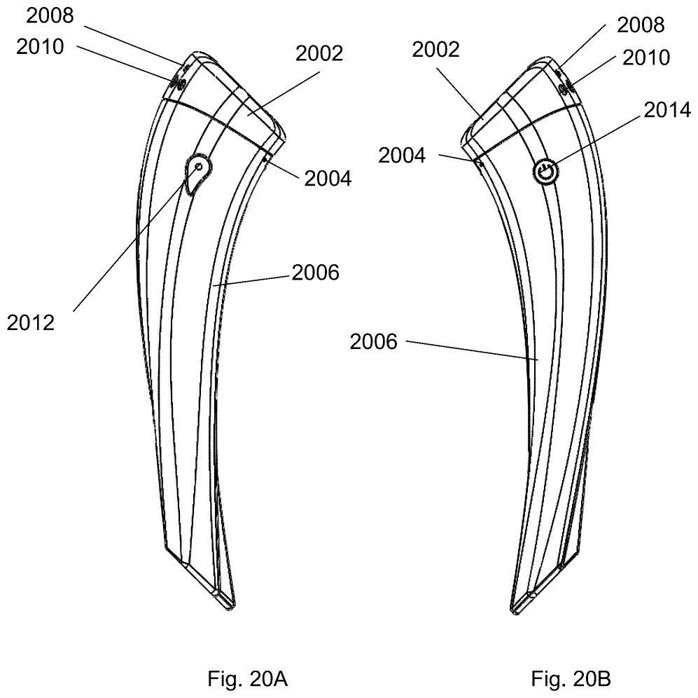

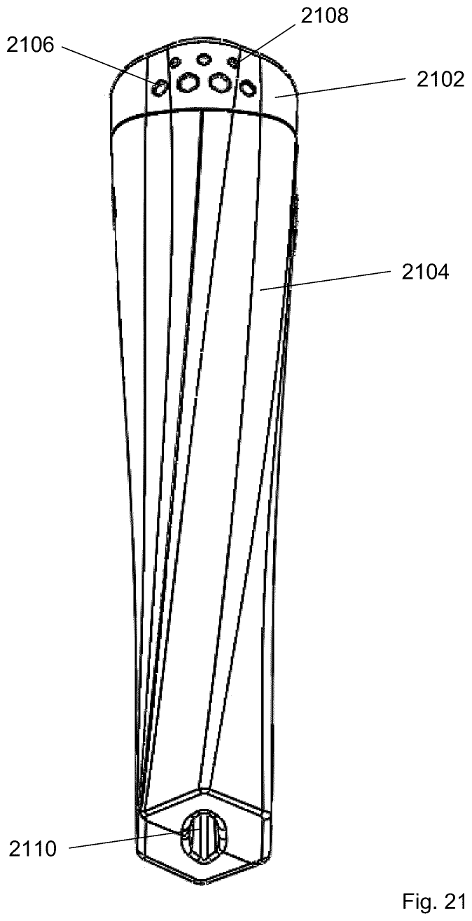

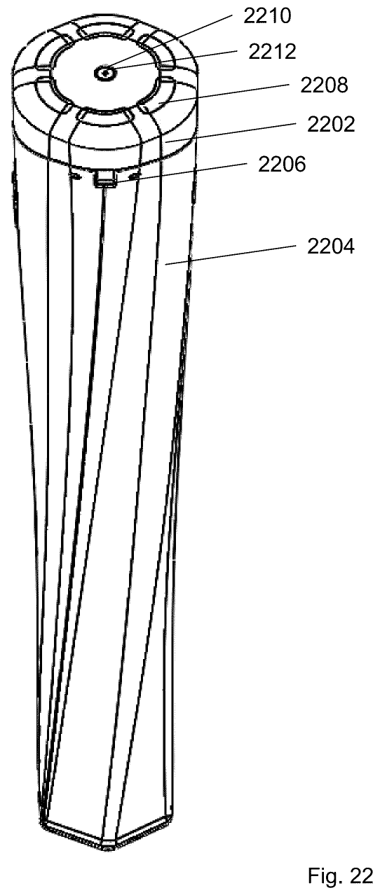

FIGS. 20A-B, FIG. 21 and FIG. 22 are schematic representations of a closed dispenser from various views in accordance with an aspect of the current invention. For example, the dispenser may include a head 2002, 2102, 2202 connected to a body 2006, 2104, 2204. Optionally, the head 2002, 2102, 2202 may be connected to the body 2006, 2104, 2204 (e.g., by a hinge 2206, 2004) in a way that allows the head 2002, 2102, 2202 to open and close. Optionally, the head may include one or more activators 2008, 2208, one or more vents 2010, 2106, and/or one or more indicator lights 2108. Optionally, the head may include one or more openings 2210 through which one or more nozzles 2212 may protrude. Optionally, the body may include a user interface, which may include a power button 2014, switch 2012, etc. Optionally, the base of the body may include a charging port 2110. In some embodiments, the head may be replaceable e.g., for different kinds and/or sizes of cartridges and/or for different numbers of nozzles.

According to some embodiments, one or more activators and/or additional treatment modules may be located on the top and/or the side of the dispenser head. Optionally, the one or more activators may be one or more lights of a selected frequency (e.g., UV, IR, etc.), one or more heaters (e.g., heating pads, flex heating elements, etc.), one or more additional activators (e.g., ultrasound, massage pads, etc.) and/or combinations thereof. Optionally, one or more vents may be located on the side of the dispenser head. Optionally, the user interface may include a power button and/or pressure switch, which may be located on the same or different parts of the dispenser body. Optionally, a charging port may be located on or within the base of the dispenser. Optionally, the base of the dispenser may include a cavity into which a charging station and/or stand may fit.

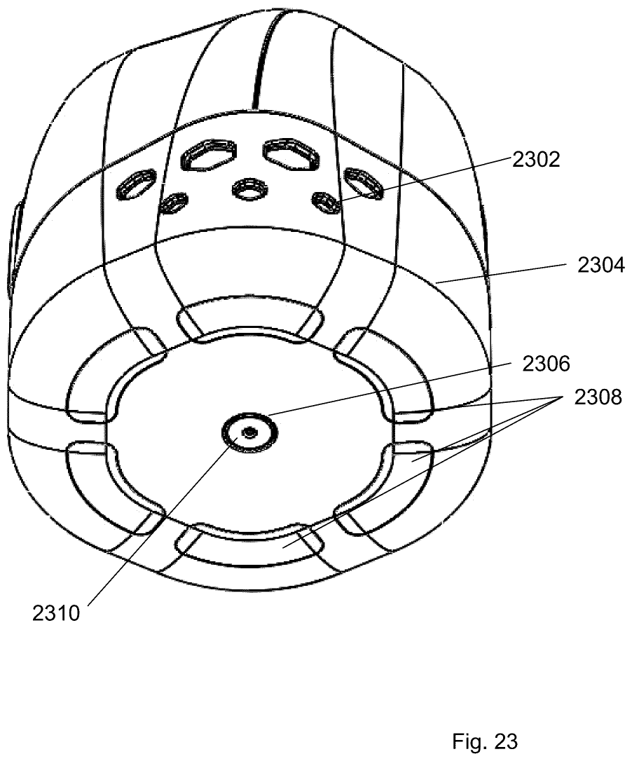

FIG. 23 is a perspective view of a single nozzle dispenser in accordance with some embodiments of the current invention. For example, the dispenser may include a head 2304 which may include one or more vents 2302 on one or more sides to release pressure and/or heat. Optionally, the top of the head may include one or more activators 2308 and an opening 2306 for a nozzle 2310.

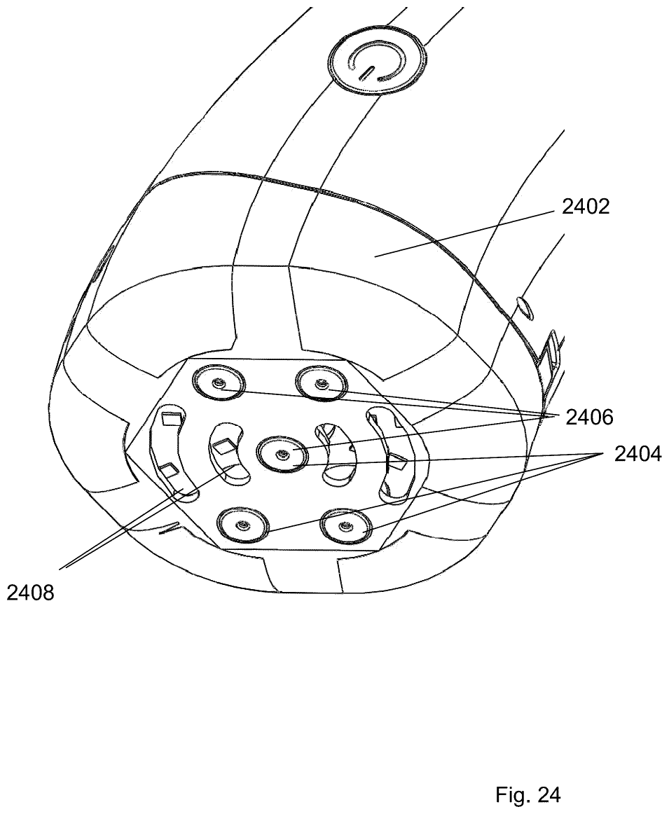

FIG. 24 is a perspective view of a multi-nozzle dispenser in accordance with some embodiments of the current invention. For example, the dispenser may include a head 2402, optionally, the top of the head may include one or more activators 2408 and multiple openings 2404 configured to accommodate one or more nozzles 2406 from a capsule (not shown) inserted into the dispenser.

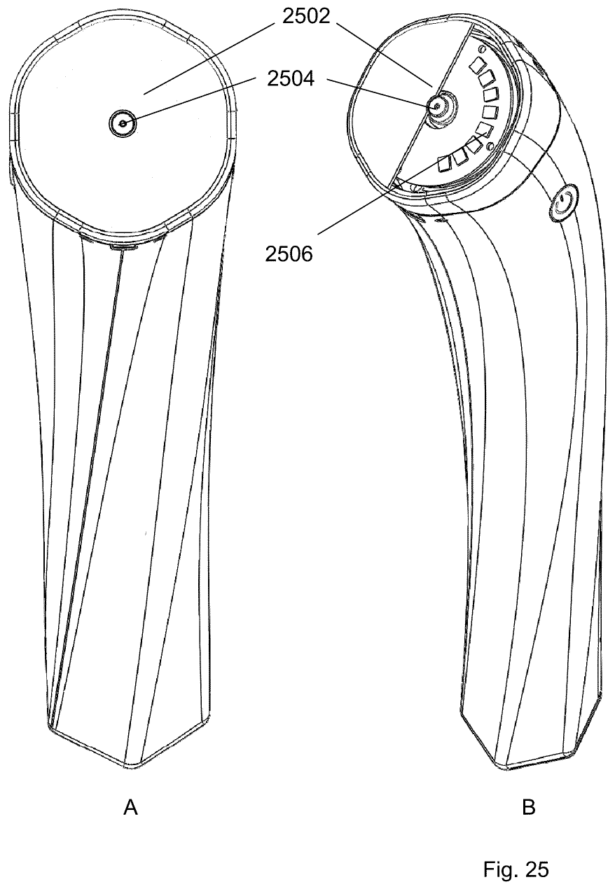

FIGS. 25A-B are perspective views of a smooth faced dispenser in accordance with some embodiments of the current invention. For example, the head 2502 of a dispenser may be smooth, e.g., without protruding and/or recessed activators. Optionally, the nozzle 2504 and/or opening for the nozzle may be flush with the outer surface of the dispenser head. Optionally, activators 2506 may be included below the smooth outer surface of the dispenser, as shown in the partial cut away view in FIG. 25B.

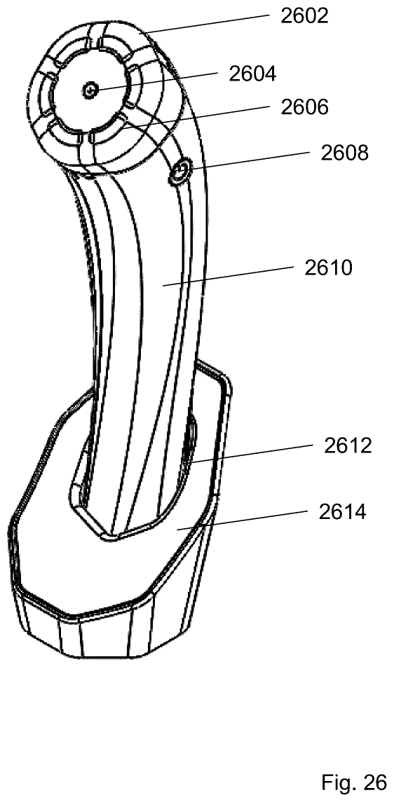

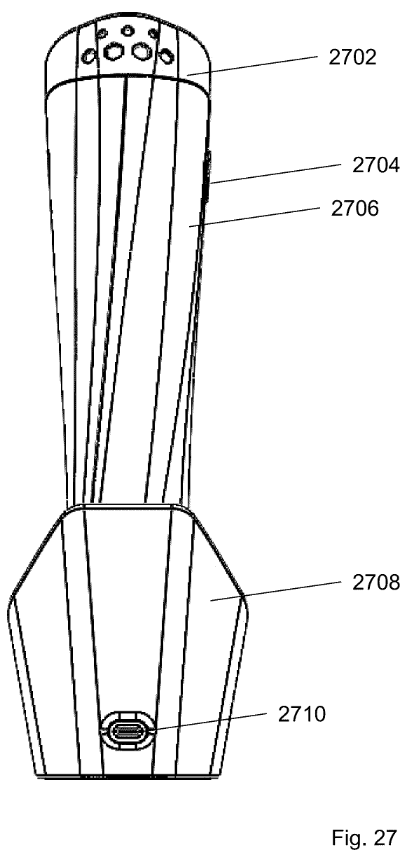

FIGS. 26-27 are schematic representations of a closed dispenser connected to a charging station from various views in accordance with an aspect of the current invention. For example, a dispenser may include a head 2602, 2702 with an opening for a nozzle 2604, and optionally, one or more activators 2606. Optionally, the head of the dispenser may be connected to the body 2610, 2706 of the dispenser, which may include a user interface 2608, 2704 the body 2610, 2706 of the dispenser may fit into a docking port 2612 of a charging station 2614, 2708. Optionally, the charging station may include a socket 2710 for connection to an electricity source. Optionally, the socket 2710 may be a USB type A port, USB type B port, USB type C port, USB mini type A port, USB mini type B port, a NEMA 1-15P port, NEMA 5-15P port, Class 1 port, Class 2 port, 2 pole port, 2 pin port, 3 pole port, 3 pin port, 2 pin port with earth, 2 pin port with ground, Schuko port, UK port, Vector apple lightning, micro and mini USB, type-c port, jack charge connector, audio wire port, Vector port, SVG, and/or EPS.

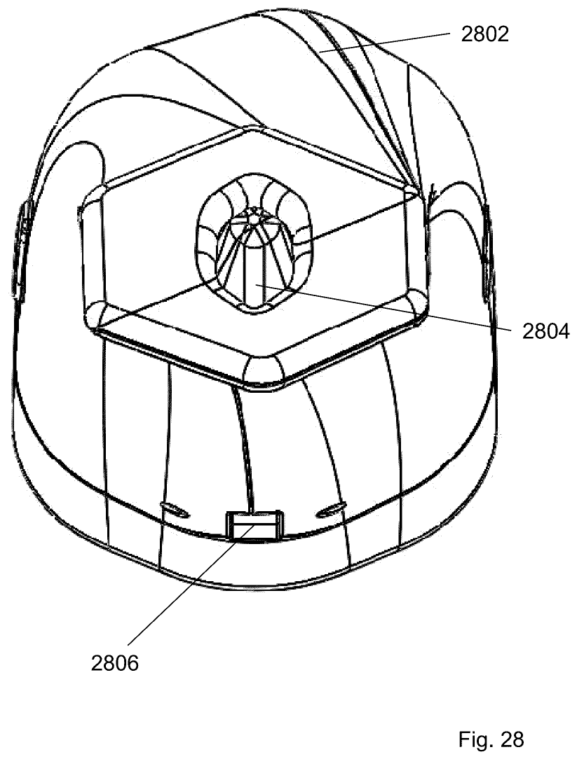

FIG. 28: Perspective view of a charging station in accordance with some embodiments of the current invention. For example, the charging station 2802 may include a port 2804 for connection to the dispenser, and a socket for connection to an electricity source 2806.

According to some embodiments, the base of the dispenser body may comprise a port for connecting it to a charging station. Optionally, the charging station may be connected by a port and/or socket to an electricity source, such as an AC or DC power source, e.g., 110 AC, 220 AC, 24V DC (e.g., an electricity grid, a car battery, photovoltaic cell, etc.) and/or with different kinds of plugs and/or sockets and/or combinations thereof. Optionally, the dispenser and/or charging station may contain one or more integrated wireless chargers. Optionally, the port may include a USB port. Optionally, the charging station may be unique to the dispenser. Optionally, the charging station may be fixed to a surface, such as a table, floor, wall, etc. Optionally, the charging station may be portable. Alternatively or additionally, the dispenser may include a plug (for example, USB type A port, USB type B port, USB type C port, USB mini type A port, USB mini type B port, a NEMA 1-15P port, NEMA 5-15P port, Class 1 port, Class 2 port, 2 pole port, 2 pin port, 3 pole port, 3 pin port, 2 pin port with earth, 2 pin port with ground, Schuko port, UK port, Vector apple lightning, micro and mini USB, type-c port, jack charge connector, audio wire port, Vector port, SVG, and/or EPS) that can be connected to a charging wire without a separate charger stand.

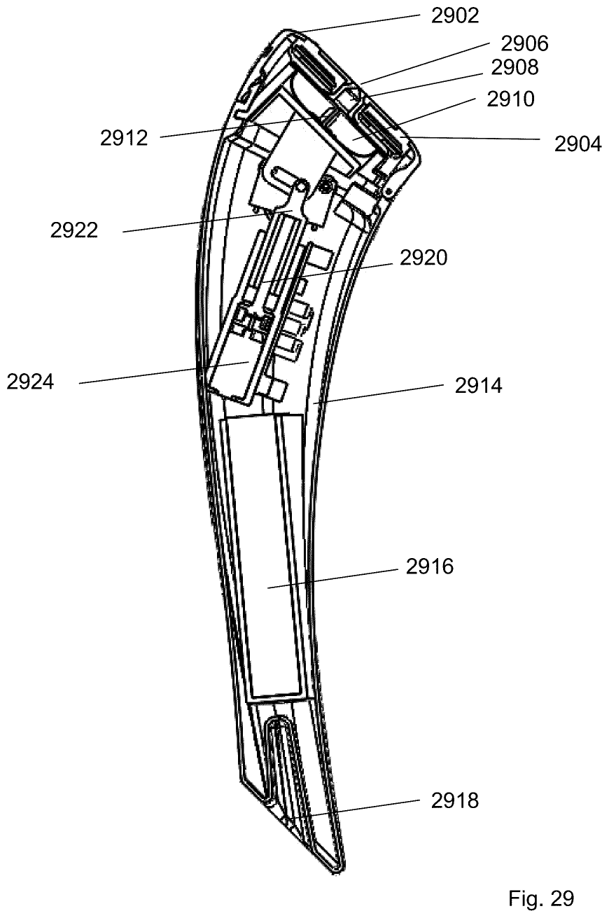

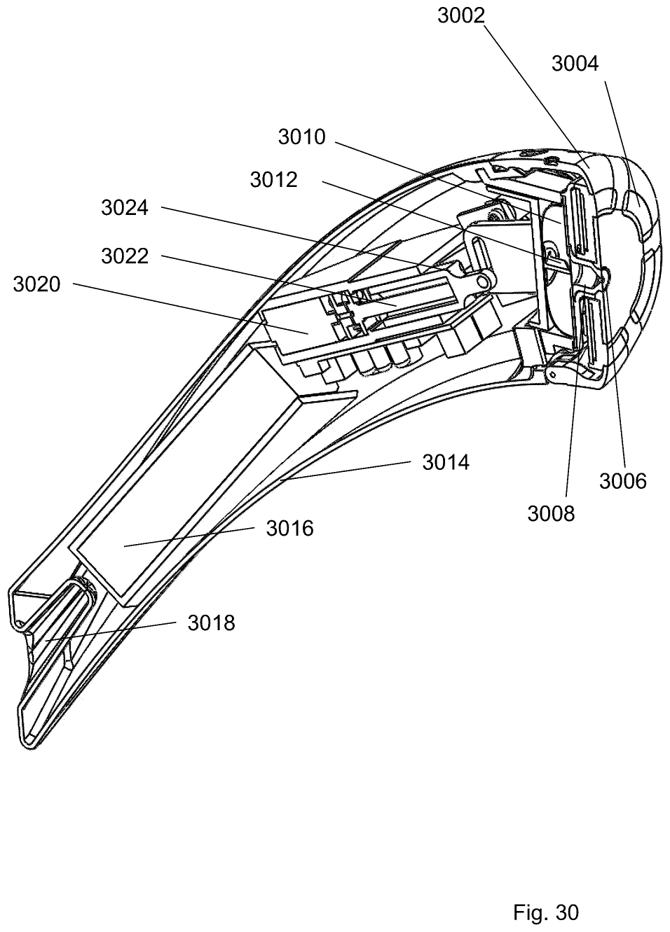

FIGS. 29 and 30 are cut away views of schematic representations of a closed dispenser in accordance with an aspect of the current invention. For example, the dispenser may include a head 2902, 3002, which may include one or more activators 2904, 3004, and one or more openings 2906, 3006 for one or more nozzles 2908, 3008 of a capsule 2910, 3010 which may include one or more punches 2912, 3012. Optionally, the body 3014, 2914 of the dispenser may include a rechargeable battery 2916, 3016, and a charging port 2918, 3018. Optionally, the dispenser body may include an actuator 2920, which may include an driver 2922 and a motor 2924, 3020. Optionally, the driver may include a ball-screw arm 3024 and/or a lead screw 3022. Optionally, the capsule may fit into an opening extending through the dispenser head. Optionally, the base of the capsule holder in the dispenser body may sit on a piston and/or driver 2922. In some embodiments, a simple rotational motor (e.g., a DC motor and/or a brushless motor) drives a piston to put linear force. For example, the linear force may squeeze a cartridge to eject its contents. Optionally, the linear pressure on the cartridge may be at an angle to the axis of rotation of the motor.

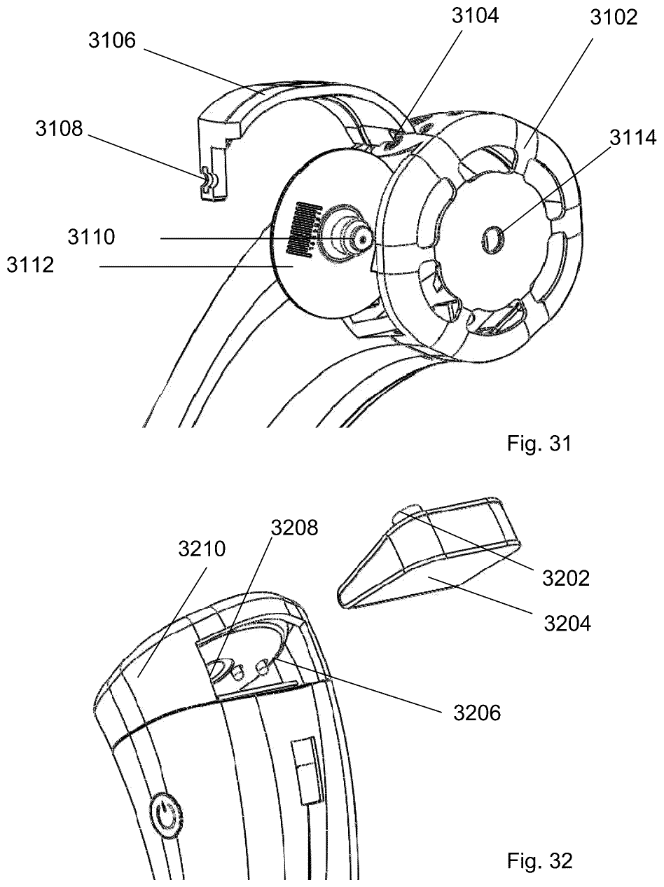

FIG. 31 is a perspective view of an open side loading dispenser with a capsule in accordance with some embodiments of the current invention. For example, the dispenser head 3102 may include a sided opening. Optionally, the opening may include a hinged 3104 door 3106. For example, the door 3106 may be locked 3108. Optionally, a capsule 3112 may be inserted into the dispenser head, such that the capsule nozzle 3110 aligns, and/or protrudes through an opening 3114 in the dispenser head 3102. For example, an accordion type cartridge (e.g., as illustrated in FIG. 19 and/or a cartridge as illustrated in FIGS. 1-7).

FIG. 32 is a perspective view of an open side loading dispenser with a capsule in accordance with some embodiments of the current invention. For example, a capsule 3204 may be inserted into a slot and/or opening 3206 in the side of the head 3210 of a dispenser such that the capsule nozzle 3202 aligns, and/or protrudes through an opening 3208 in the dispenser head 3210. For example, a book type cartridge (e.g., as illustrated in FIGS. 13 and 14.

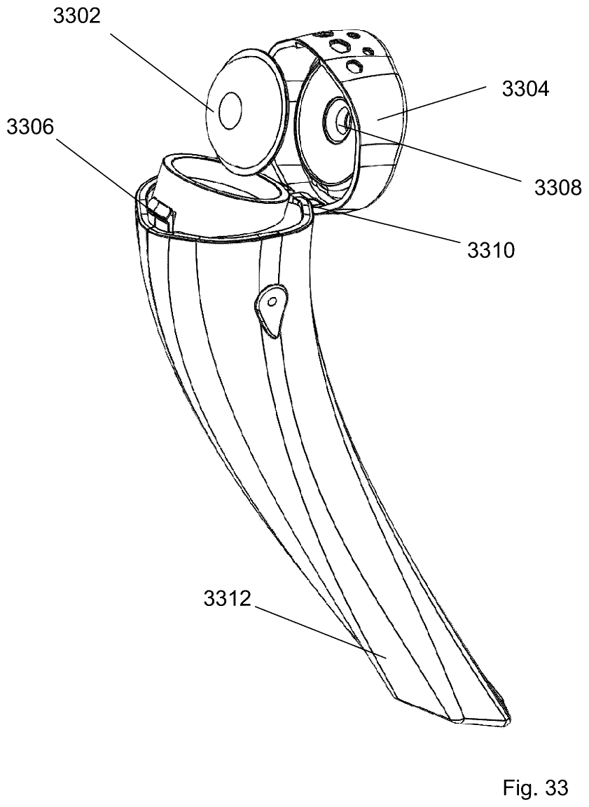

FIG. 33 is a perspective view of an open dispenser and capsule in accordance with some embodiments of the current invention. For example, a capsule 3302 may be inserted into a dispenser, wherein the head 3304 of the dispenser may be connected to the body 3312 of the dispenser e.g., by a hinge 3310. and which may be locked closed by a lock 3306. The capsule 3302 may be located within the dispenser such that a nozzle (not shown) of the capsule 3302 may align with and/or protrude through an opening 3308 in the dispenser head 3304.

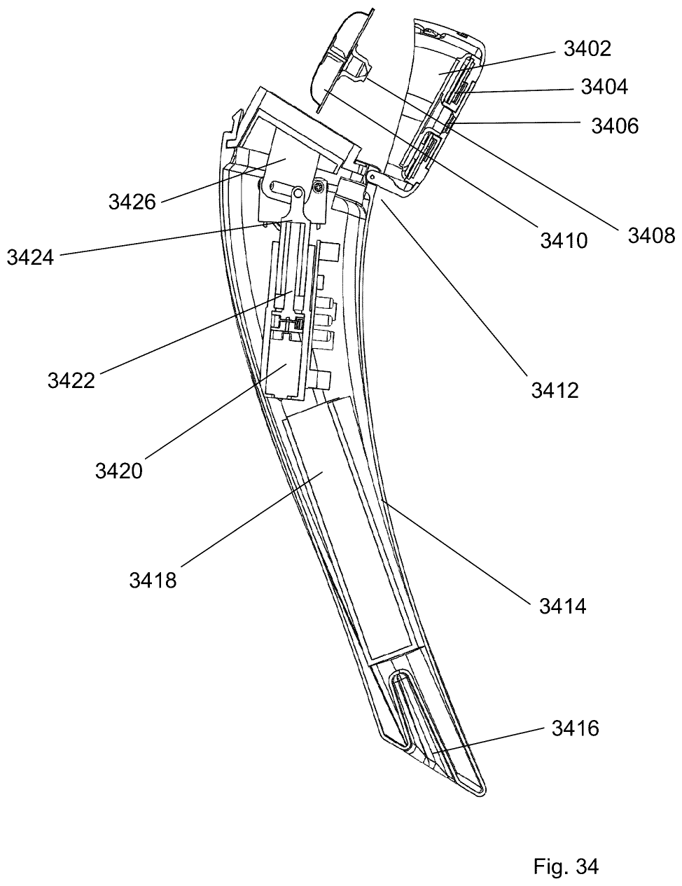

FIG. 34 is a cut away view of an open dispenser with a round capsule in accordance with some embodiments of the current invention. For example, a dispenser may be opened such that the head 3402, which may include one or more activators 3404 and at least one opening 3406 for a nozzle 3408 of a capsule 3410. Optionally, the head 3402 may be connected to the body 3414 of the dispenser e.g., by a hinge 3412. Optionally, the body 3414 may include a charging port 3416 connected to a rechargeable battery 3418, which may run an actuator comprising a motor 3420 attached to a lead screw 3422, connected to a ball-screw arm 3424, which may drive a plunger 3426. Optionally, one or more batteries 3418 may be located below the actuator. Optionally, the actuator may be sealed off from the capsule, e.g., to prevent contamination of the dispenser.

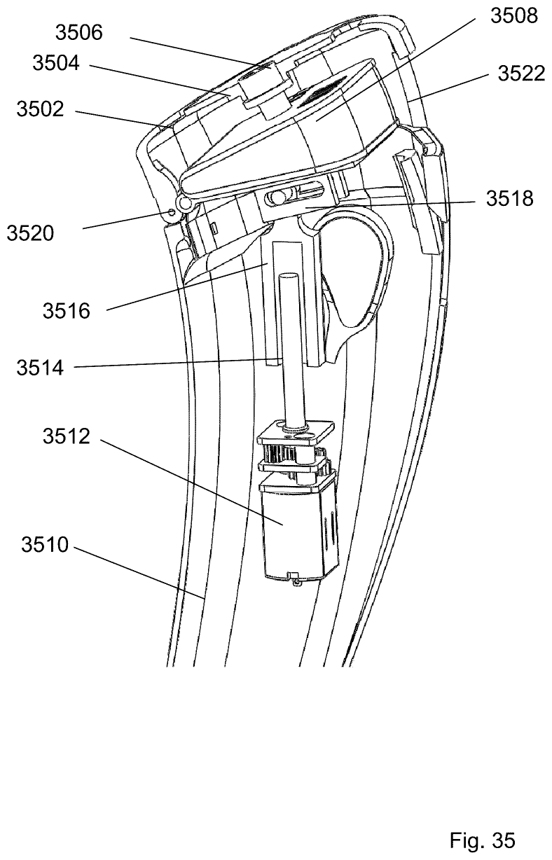

FIG. 35 is a cut away view of an open dispenser with a book capsule in accordance with some embodiments of the current invention. For example, a dispenser may be opened such that the head 3502, which may include a side opening 3522 and one or more activators and at least one opening 3504 for a nozzle 3506 of a capsule 3508. Optionally, the head 3502 may be connected to the body 3510 of the dispenser e.g., by a hinge 3520. Optionally, the body 3510 may include an actuator comprising a motor 3512 attached to a lead screw 3514, connected to a ball-screw arm 3516, which may drive a plunger 3518. Optionally, the plunger 3518 may press the capsule 3508 against the inner walls of the head 3502 to squeeze the ointment out of the capsule 3508 through the nozzle 3506.

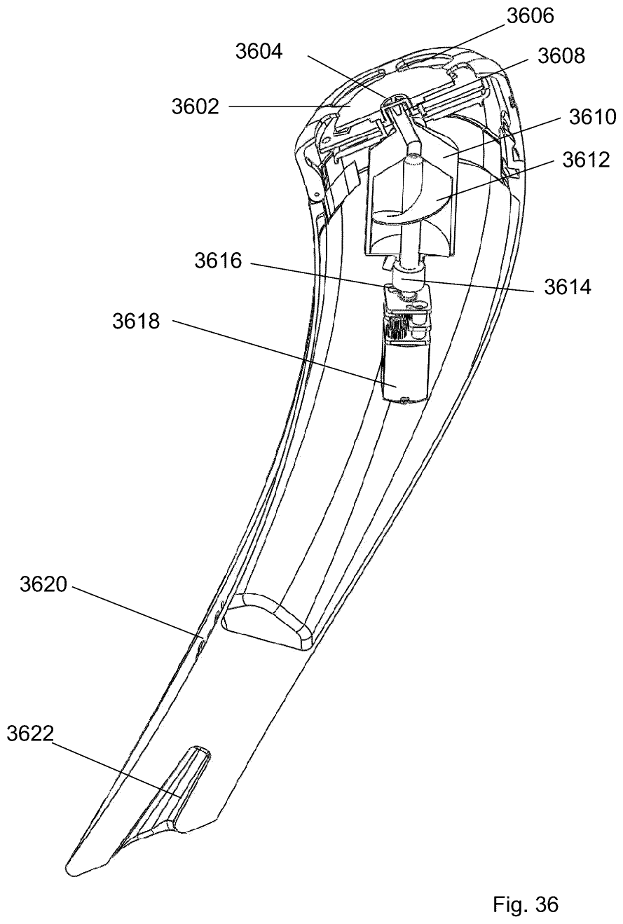

FIG. 36 is a cut away view of an open dispenser with a screw capsule in accordance with some embodiments of the current invention. For example, a dispenser may include a head 3602, which may include one or more activators 3606 and at least one opening 3604 for a nozzle 3608 of a capsule 3610. Optionally, the capsule 3610 may include a screw 3612, which may be connected by a screw attachment 3614 be connected to a lead screw 3616 which may be rotated by a motor 3618 within the body 3620 of the dispenser. Optionally, the body 3620 may include a charging port 3622 which may be connected to a rechargeable battery (not shown). Optionally, rotation of the screw may push the ointment out of the nozzle of the capsule.

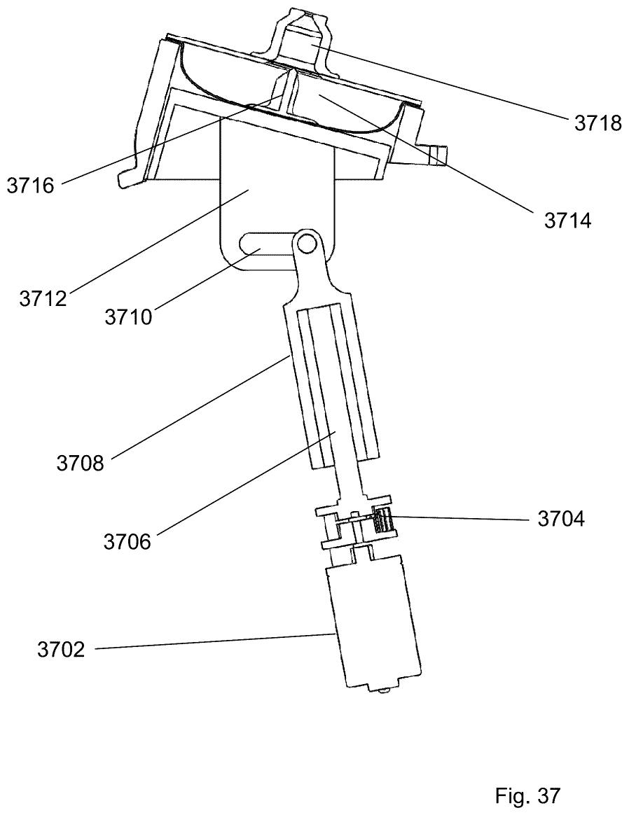

FIG. 37 is a perspective view of a schematic representation of a driver in accordance with an aspect of the current invention. For example, the driver may include a piston arm. Optionally, an actuator (e.g., a motor) 3702 may be connected through a transmission 3704 to a lead screw 3706, a ball-screw arm 3708 to a slide track 3710 attached to a plunger 3712. Optionally, the plunger 3712 may press the punch 3716 of the capsule 3714 through the top of the capsule 3714 and to dispense the ointment through the nozzle 3718.

According to some embodiments, the driver may be operated manually by the user in order to apply the desired pressure to dispense the required amount of ointment. Optionally, the piston may be located on the upper portion of the dispenser body, and it may squeeze the capsule against the inner wall of the dispenser head. Optionally, the driver may raise a floor against the base of the capsule while simultaneously lowering a floor onto the top of the capsule, thereby squeezing the capsule from two directions at the same time. Optionally, the driver may apply pressure on the capsule from one or multiple directions at the same time.

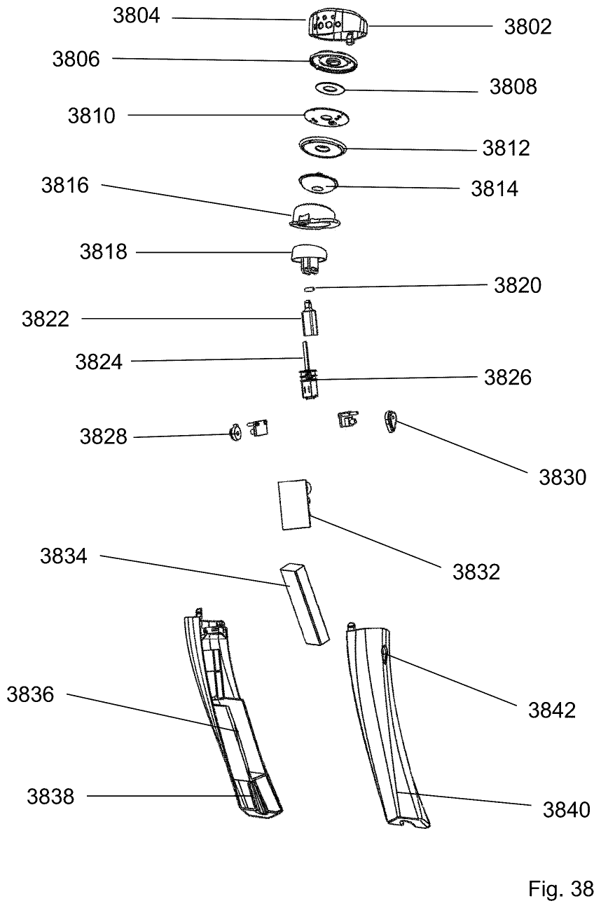

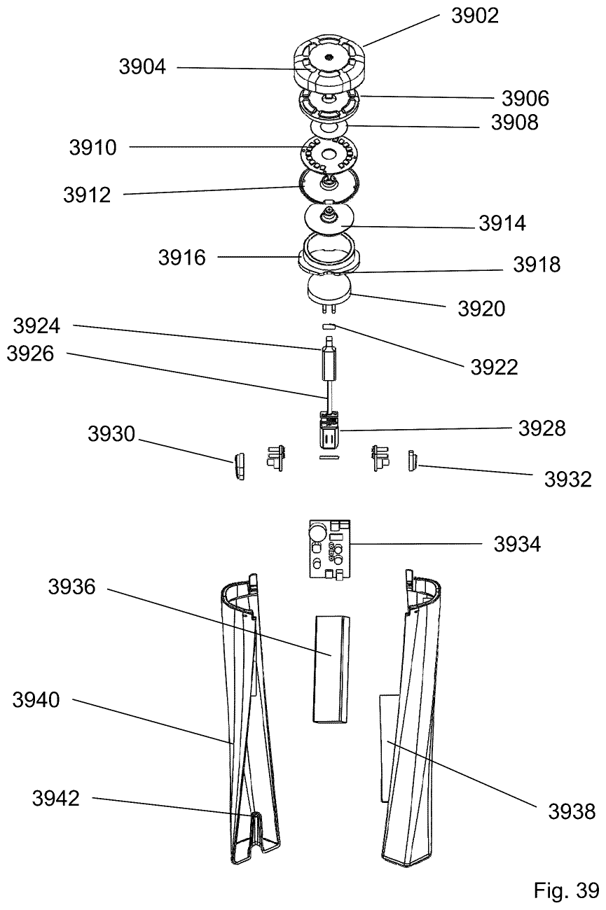

FIGS. 38 and 39 are exploded schematic representations of a dispenser and capsule from various views in accordance with an aspect of the current invention. For example, the dispenser may include a head 3802, 3902 which may include one or more vents 3804, one or more activators 3904, one or more light guides 3806, 3906, a heating element 3808, 3908, one or more LED PCB 3810, 3910, and a capsule retainer 3812, 3912 (e.g., to hold a capsule 3814, 3914 in place). Optionally the dispenser head 3802, 3902 may be connected to a dispenser body 3840, 3940. Optionally, the body 3840, 3940 may include a capsule holder 3816, 3916, optionally with a lock 3918, which may be connected to a plunger 3818, 3920, a slider track or shaft 3820, 3922 attached to a ball-screw arm 3924, 3822 linked to a lead screw 3824, 3926 driven by a motor 3826, 3928 connected to a power button 3828, 3930 and a switch 3830, 3932 (which may control the pressure applied to the capsule, activate one or more activators, etc.) which may be controlled by a processor 3832, 3934 powered by one or more batteries 3834, 3936, which may rest in a battery housing 3836, 3938 which may be connected to a charging port 3838, 3942. Optionally, the body may include one or more holes or openings 3842 for user interface buttons, e.g., a power button, switch, etc.

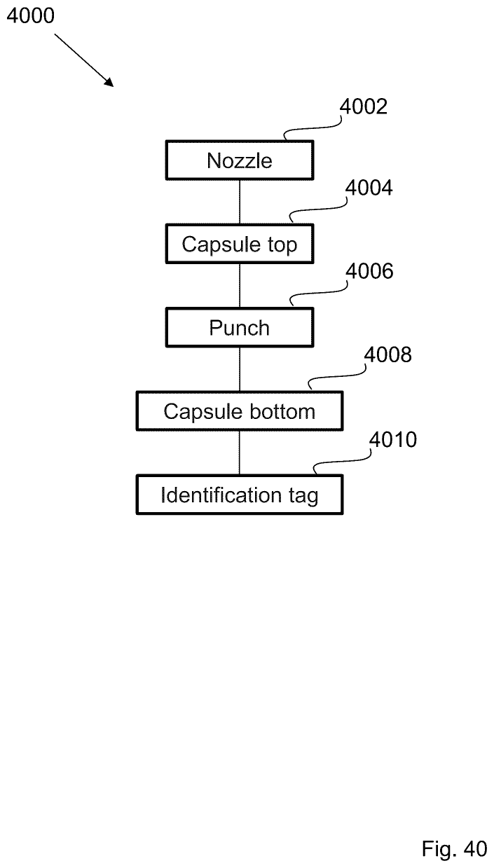

FIG. 40 is a block diagram of a capsule in accordance with some embodiments of the current invention. For example, the capsule 4000 may include a nozzle 4002 attached to a capsule top 4004 sealed to a capsule bottom 4008 including a punch 4006, wherein the capsule 4000 may include an identification tag 4010.

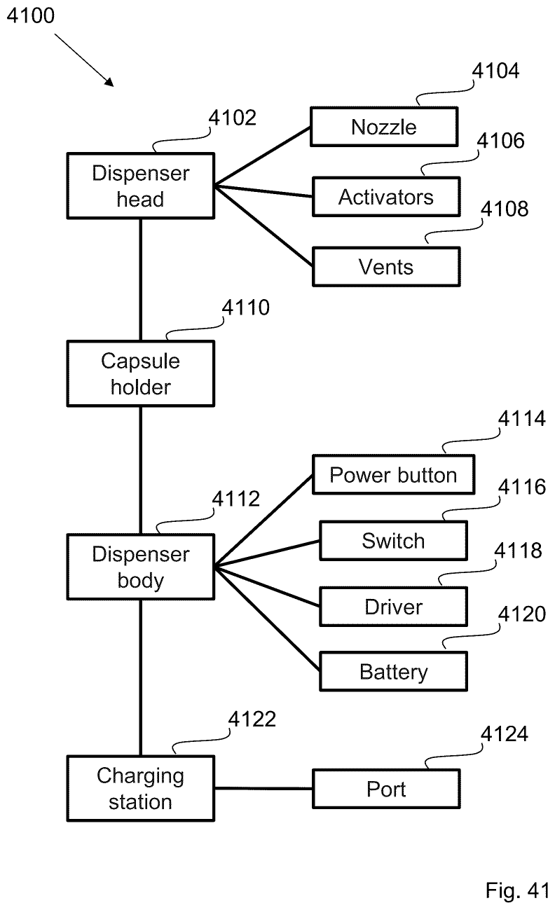

FIG. 41 is a block diagram of a dispenser in accordance with some embodiments of the current invention. For example, a dispenser 4100 may include a dispenser head 4102 including a nozzle housing 4104, one or more activators 4106, one or more vents 4108, and optionally one or more guide lights. Optionally, the dispenser head and dispenser body may together provide a sealed capsule holder 4110. Optionally, the dispenser body 4112 may include a user interface, such as a power button 4114, a switch 4116, etc., an driver 4118, and a rechargeable battery 4120, and a charging port which may be connected to a charging station 4122 with a socket 4124 for connection to an electricity source.

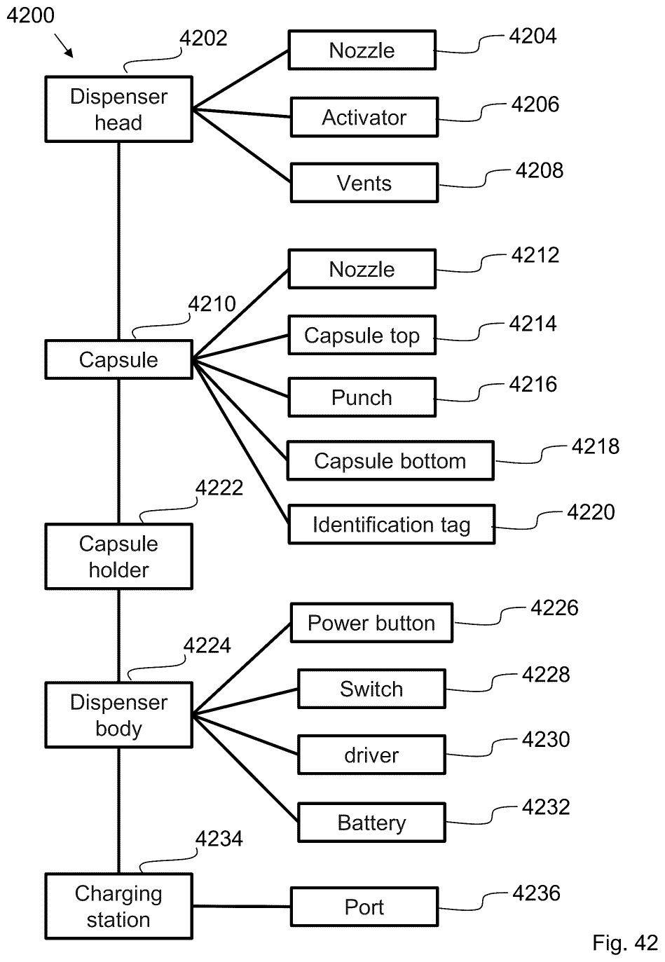

FIG. 42 is a block diagram of a capsule in a dispenser in accordance with some embodiments of the current invention. For example, a dispenser 4200 may include a dispenser head 4202 including a nozzle housing 4204, one or more activators 4206, one or more vents 4208, and optionally one or more guide lights. Optionally, the dispenser head and dispenser body may together provide a sealed capsule holder 4222 for a capsule 4210. Optionally, the capsule 4210 may include a nozzle 4212 attached to a capsule top 4214 sealed to a capsule bottom 4218 including a punch 4216, wherein the capsule 4000 may include an identification tag 4220. Optionally, the dispenser body 4224 may include a user interface, such as a power button 4226, a switch 4228, etc., an driver 4230, and a rechargeable battery 4232, and a charging port which may be connected to a charging station 4234 with a socket 4236 for connection to an electricity source.

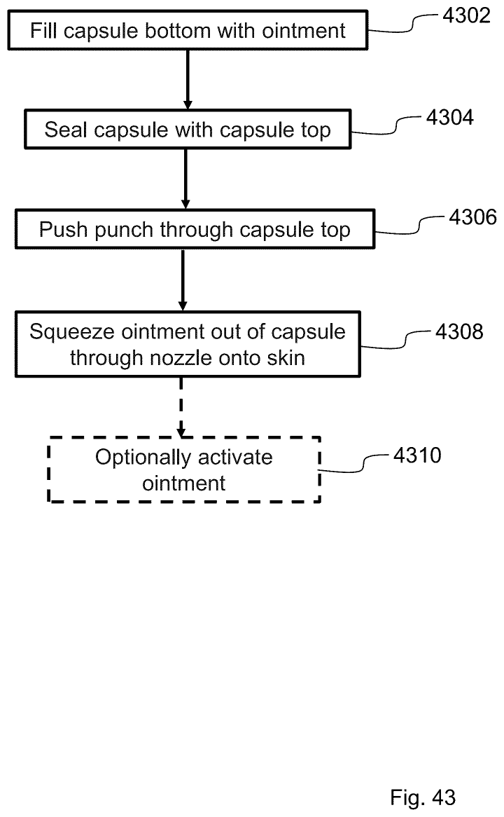

FIG. 43 is a flow diagram describing the use of the capsule in accordance with an embodiment of the current invention. Optionally, the capsule may be used with a dedicated dispenser. Optionally, the capsule may be used without a dedicated dispenser. Optionally, a capsule may be used by hand, e.g., manually squeezing the capsule. Optionally, if a capsule is used without a dedicated dispenser, the ointment may be activated by an additional device and/or a dedicated dispenser.

For example, a capsule bottom is filled 4302 with an ointment. Optionally, the capsule bottom is scaled 4304 with a capsule top. Optionally, the capsule is either placed in a dispenser or used by hand. Optionally, the punch is pushed 4306 through the capsule top. Optionally, the ointment is squeezed out 4308 of the capsule onto the skin. Optionally, the ointment is activated 4310, before, during or after application.

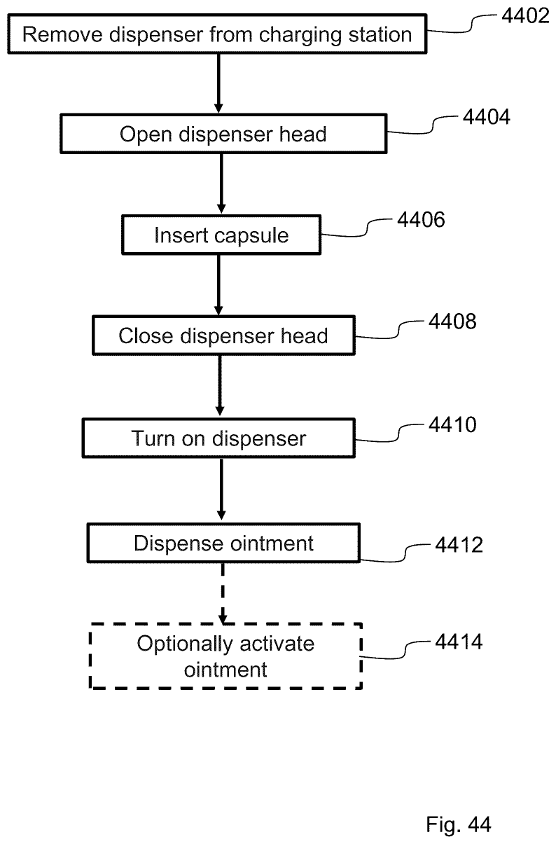

FIG. 44 is a flow diagram describing the use of the dispenser in accordance with an embodiment of the current invention.

For example, a dispenser is removed 4402 from a charging station. The dispenser head is opened 4404. A capsule is inserted 4406 into the dispenser. The dispenser head is then closed 4408 and locked. The dispenser is turned on 4410. The ointment is dispensed 4412 and/or activated 4414 by the dispenser.

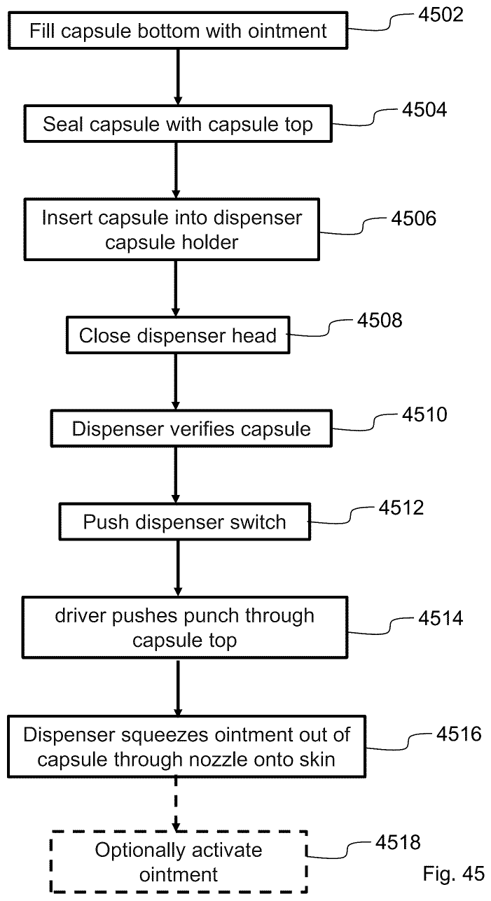

FIG. 45 is a flow diagram describing the use of the dispenser in accordance with an embodiment of the current invention. For example, a capsule bottom is filled 4502 with an ointment. The capsule is then sealed 4504 with a capsule top. A dispenser is taken from a charging station and the dispenser head is opened. The capsule is inserted 4506 into a dispenser capsule holder. The dispenser head is then closed 4508 and locked. The dispenser is turned on. The dispenser then verifies 4510 if the capsule is genuine. Once verified, the driver is triggered 4512. The driver pushes 4514 the punch through the capsule top, and then squeezes 4516 the ointment out of the capsule through the nozzle onto the skin. Optionally, the ointment is activated 4518, before, during or after application.

For example, a capsule bottom may be filled with an ointment, the capsule may then be sealed. The capsule may then be stored for an extended period without affecting the stability of the ointment. When required, a user may then load a capsule into a dispenser. The user may then turn on the dispenser and dispense the ointment directly onto the skin. The ointment may optionally be activated. The capsule may then be replaced by a capsule comprising a different ointment for a different use or for use on a different part of the body.

These embodiments are provided by way of example and are in no means intended to be limiting the scope of the invention.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

### General

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

It is expected that during the life of a patent maturing from this application many relevant building technologies, artificial intelligence methodologies, computer user interfaces, image capture devices will be developed and the scope of the terms for design elements, analysis routines, user devices is intended to include all such new technologies a priori.

Unless otherwise defined, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein the term “about” refers to +10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-29. (canceled)

30. An ointment capsule comprising:
an identification tag;
at least one nozzle attached to or integrated with a capsule top;
the capsule top sealed to a capsule base; and
at least one punch aligned with the at least one nozzle, wherein the punch is configured to penetrate the capsule top and dispense the ointment through the nozzle when pressure is applied to the capsule.

31. The capsule according to claim 30, wherein the capsule is round, cylindrical, bowl shaped, circular, book shaped, accordion shaped, bottle shaped, square shaped, cubical, or pyramidal in shape.

32. The capsule according to claim 30, wherein a width of the capsule ranges between about 1 mm to about 5 cm.

33. The capsule according to claim 30, wherein the nozzle has a length of between about 1 mm to about 1 cm.

34. The capsule according to claim 30, wherein the punch has a sharp tip.

35. The capsule according to claim 30, wherein the punch does not completely occlude the nozzle on penetration of the capsule top.

36. The capsule according to claim 30, wherein the capsule includes a screw.

37. The capsule according to claim 30, wherein the identification tag is selected from the group consisting of a barcode, QR code, RFID, magnetic code, or a readable chip.

38. An ointment dispenser comprising:
a dispenser head, wherein the dispenser head comprising at least one nozzle opening and at least one activator;
a capsule holder fitted within a dispenser body, wherein the dispenser head and the dispenser body are connected to each other; and
a driver located within the dispenser body configured to push the capsule holder against an inner surface of the dispenser head.

39. The dispenser according to claim 38, wherein the at least one activator is selected from a group consisting of a light of a particular frequency, a heater, ultrasound, and a combination thereof.

40. The dispenser according to claim 38, wherein the dispenser head further comprises a slot into which a capsule is inserted.

41. The dispenser according to claim 38, wherein the dispenser body further comprises a rechargeable battery and a battery housing.

42. The dispenser according to claim 38, wherein the driver comprises a piston arm connected through a transmission to a lead screw and a ball-screw arm attached to a slide track on a plunger.

43. The dispenser according to claim 38, wherein the dispenser further comprises an identification tag reader.

44. The dispenser according to claim 43, wherein the identification tag reader is configured to verify a veracity of a capsule inserted into the dispenser.

45. A method for dispensing an ointment from an ointment capsule using an ointment dispenser, the method comprising:
inserting an ointment capsule into a capsule holder in an ointment dispenser;
verifying the capsule;
triggering a driver in a body of the dispenser to compress the verified capsule;
pushing a punch through a capsule top; and
squeezing the ointment out of a nozzle of the verified capsule through a head of the dispenser.

46. The method according to claim 45, wherein the verifying comprises reading an identification tag on the capsule and verifying the read identification tag using an internal database.

47. The method according to claim 45, wherein the verifying comprises reading an identification tag on the capsule and verifying the read identification tag by connecting to an on-line database over a wireless network.

48. The method according to claim 45, further comprising activating the ointment using at least one activator on the head of the dispenser, wherein the at least one activator is selected from a group consisting of a light of a particular frequency, a heater, ultrasound, and a combination thereof.

49. The method according to claim 48, wherein the activating takes place before, during or after application of the ointment.
